(19) United States
(12) Patent Application Publication
(10) Pub. No.: US 2026/0368519 A1
(43) Pub. Date: Nov. 19, 2026

(54) MESSAGING DEVICE, METHOD AND PROGRAM

(71) Applicant: NTT, Inc., Tokyo (JP)

(72) Inventors: Shuhei YAMAMOTO, Musashino-shi (JP); Takeshi KURASHIMA, Musashino-shi (JP); Yasunori AKAGI, Musashino-shi (JP); Yoshiaki TAKIMOTO, Musashino-shi (JP); Tomu TOMINAGA, Musashino-shi (JP)

(73) Assignee: NTT, Inc., Tokyo (JP)

(21) Appl. No.: 19/475,439

(22) PCT Filed: Apr. 19, 2023
(86) PCT No.: PCT/JP2023/015624

§ 371 Date: Oct. 15, 2025

(51) Int. Cl. G06N 3/08; G16H 20/00

(52) U.S. Cl. CPC G06N 3/08; G16H 20/00

(57) ABSTRACT

An embodiment includes first processing circuitry that analyzes a present bias of a target user, and second processing circuitry that performs messaging to the target user. The first processing circuitry trains a present bias analysis model on the basis of feature data generated from vital data, action data, and task data of a plurality of learning target users, and the second processing circuitry trains a message classification model by using a token list of a plurality of pieces of training message data and label data.

### TECHNICAL FIELD

One aspect of the present invention relates to a messaging device, method and program for messaging a user in consideration of a present bias.

### BACKGROUND ART

Health conditions of people tend to deteriorate due to a life of insufficient exercise or oversitting. According to some investigations, the proportion of obese patients and diabetic patients is increasing year by year in Japan, and simultaneously the proportion of people having exercise habits is decreasing. The proportion of home workers has increased in Japan after the year 2020, and this trend is expected to continue to grow in the future. In order to improve and maintain the healthy condition of an individual, it is necessary to promote proper exercise and health diagnosis.

Therefore, methods of inducing human actions by intervention using messaging have been proposed. For example, a technique using framing has been proposed as one of the methods. Framing emphasizes some representations in a message, and emphasizes benefits obtained by achieving actions described in a message or losses received by not acting (refer to Non Patent Literature 1, for example). However, since the messaging method using framing does not take into account personal characteristics, the effect is likely to be limited.

On the other hand, as a method of indexing personal characteristics, an indicator known as a present bias has attracted attention in behavioral economics. The present bias is a cognitive bias that leads to overestimating patience in the distant future and becoming impatient in the near future, that is, a human psychological characteristic in which a large benefit that can be obtained in the future is weighed against a small benefit that can be obtained immediately and the latter is selected. People with a strong present bias tend to succumb to short-term temptations and tend to put off carrying out their plans. As a result, it is known that people with a strong present bias are less likely to undergo health checkups due to their aversion to immediate hardships, and are more likely to have lifestyle habits such as those causing obesity and smoking (refer to Non Patent Literature 2, for example).

As a method of estimating a present bias, for example, a method of estimating a present bias using a questionnaire has been proposed. This method is an estimation method using a “selection type questionnaire”, and for example, a plurality of questions such as “Would you rather receive X yen today (option A) or Y yen 7 days later (option B)?” are prepared with Y yen being higher than X yen in the two options, and respondents are able to make a selection. Here, by making the amount of money of option B higher, the question designer is creating an “annual interest” like a bank deposit. Regarding the response results, a question in which selection has been switched from option A (receiving today) to option B (receiving seven days later) is noted, and on the assumption that a discount rate of a respondent lies between the annual interest in the question in which selection has been switched and the annual interest in a question immediately preceding the question, the mean of these two annual interests is adopted as a time discount rate. Similar questions are set for farther in the future (for example, 90 days and 97 days in the future) and responses are asked for, and a time discount rate is measured in a similar manner. Here, the present bias strength can be measured by comparing time discount rates between today and 7 days from now and between 90 days from now and 97 days from now (refer to Non Patent Literature 3, for example).

### CITATION LIST

### Non Patent Literature

[NPL 1] Altmann, S. and Traxler, C., “Nudges at the dentist”, European Economic Review, Vol. 72, pp. 19-38 (2014).
[NPL 2] Mischel, Walter et al. “Willpower over the life span: decomposing self-regulation”. Social cognitive and affective neuroscience, Vol. 6, No. 2 (2011): 252-6.
[NPL 3] Hardisty, David J. et al. “How to measure time preferences: An experimental comparison of three methods”. Judgment and Decision Making Vol. 8 (2013): 236-249.

### SUMMARY OF INVENTION

### Technical Problem

However, in the estimation method using selection type questionnaire, since a time discount rate of a respondent is allocated from an annual interest determined by a question designer in advance, answers are less likely to diverge, and it is necessary to answer to a plurality of questions. Further, in order to avoid respondents responding randomly (for example, continually selecting option B without consideration), the order of questions is switched at random depending on the annual interest, and thus a plurality of points where options A and B are switched between are detected, and as a result, there are many respondents to be handled as giving invalid answers. Further, the load on respondents is large since answers to a plurality of questions are required, and since the questionnaires are carried out at medium to long-term intervals, fine changes are difficult to detect. Therefore, even if the present bias of an individual estimated using selection type questionnaire is applied to messaging, effective intervention cannot be expected.

The present invention has been made in view of the above circumstances, and an object of the present invention is to provide a technology for enabling highly effective messaging by accurately estimating an individual present bias.

### Solution to Problem

To achieve the aforementioned object, a first aspect of a messaging device according to the present invention includes a present bias analysis unit configured to analyze a present bias of a target user, and a messaging controller configured to perform messaging to the target user.

The present bias analysis unit includes: a first present bias learning processing unit configured to acquire vital data, action data, and task data of a plurality of learning target users over a predetermined period, and to perform first preprocessing on the acquired vital data, action data, and task data; a second present bias learning processing unit configured to generate feature data relating to the plurality of learning target users by combining the preprocessed vital data, action data, and task data for the same learning target user and with positions being aligned in time series; and a third present bias learning processing unit configured to train a present bias analysis model having a neural network structure constructed in advance by using the feature data relating to the plurality of learning target users and present bias data that is a correct answer to generate a trained present bias analysis model.

The messaging controller includes: a first message learning processing unit configured to acquire a plurality of pieces of training message data, and to perform second preprocessing on the acquired training message data to generate a token list constituting the training message data; and a second message learning processing unit configured to train a message classification model having a neural network structure constructed in advance by using the token list of the plurality of pieces of training message data and preset label data to generate a trained message classification model.

According to the first aspect of the present invention, it is possible to create a present bias analysis model that enables estimation of a present bias of a target user using vital data, action data, and task data acquired by a vital sensor and a scheduler in the daily life of the estimation target user without relying on questionnaire surveys. In addition, by training a neural network using a plurality of pieces of training message data and label data thereof, it is possible to automatically create a message classification model for classifying messages to be presented to a target user.

In a messaging device according to a second aspect of the present invention, the present bias analysis unit further includes: a first present bias estimation processing unit configured to acquire a vital data series, an action data series, and a task data series relating to the target user; a second present bias estimation processing unit configured to perform the first preprocessing on the acquired vital data series, action data series, and task data series; a third present bias estimation processing unit configured to generate feature data relating to the target user by combining the vital data series, the action data series, and the task data series on which the first preprocessing has been performed with positions being aligned in time series; and a fourth present bias estimation processing unit configured to input the feature data relating to the target user to the present bias analysis model and to obtain present bias estimation data of the target user from the present bias analysis model.

In addition, the messaging controller further includes: a first message selection processing unit configured to acquire a plurality of pieces of presentation message data, to perform the second preprocessing on the acquired presentation message data, and to generate a token list constituting the presentation message data; a second message selection processing unit configured to input the generated token list to the message classification model and to obtain data representing rewards and types of target actions corresponding to the presentation message data from the message classification model; a third message selection processing unit configured to acquire data representing a type of a target action being executed or scheduled to be executed by the target user, and to select the presentation message data corresponding to the acquired type of the target action and having a type of reward corresponding to the present bias estimation data; and a fourth message selection processing unit configured to output the selected presentation message data to present the selected presentation message data to the target user.

According to the second aspect of the present invention, the present bias of a target user can be estimated with high accuracy, and by using the result of estimation of the present bias, it is possible to select and present message data that reflects the target user's personal personality.

### Advantageous Effects of Invention

That is, according to one aspect of the present invention, it is possible to provide a technology for enabling highly effective messaging by accurately estimating an individual present bias.

### BRIEF DESCRIPTION OF DRAWINGS

FIG. 1 is a diagram illustrating a configuration of a system including a messaging device according to an embodiment of the present invention.

FIG. 2 is a block diagram illustrating an example of a hardware configuration of the messaging device according to one embodiment of the present invention.

FIG. 3 is a block diagram illustrating an example of a software configuration of the messaging device according to one embodiment of the present invention.

FIG. 4 is a block diagram illustrating an example of configurations of a present bias analysis unit and a present bias analysis data storage unit included in the messaging device illustrated in FIG. 3.

FIG. 5 is a block diagram illustrating an example of configurations of a messaging control unit and a messaging control data storage unit included in the messaging device illustrated in FIG. 3.

FIG. 6 is a flowchart illustrating an example of an overall processing procedure and processing details of present bias analysis model training processing executed by the present bias analysis unit illustrated in FIG. 4 in a learning phase.

FIG. 7 is a flowchart illustrating an example of a processing procedure and processing details of preprocessing for vital data in the overall processing procedure illustrated FIG. 6.

FIG. 8 is a flowchart illustrating an example of a processing procedure and processing details of preprocessing for action data in the overall processing procedure illustrated in FIG. 6.

FIG. 9 is a flowchart illustrating an example of a processing procedure and processing details of preprocessing for task data in the overall processing procedure illustrated in FIG. 6.

FIG. 10 is a flowchart illustrating an example of a processing procedure and processing details of data combining processing in the overall processing procedure illustrated in FIG. 6.

FIG. 11 is a flowchart illustrating an example of a processing procedure and processing details of time information extraction processing in the overall processing procedure illustrated in FIG. 6.

FIG. 12 is a flowchart illustrating an example of a processing procedure and processing details of present bias analysis model training processing in the overall processing procedure illustrated in FIG. 6.

FIG. 13 is a flowchart illustrating an example of an overall processing procedure and processing details of message classification model training processing executed by the messaging control unit illustrated in FIG. 5 in a learning phase.

FIG. 14 is a flowchart illustrating an example of a processing procedure and processing details of message preprocessing in the processing procedure illustrated in FIG. 13.

FIG. 15 is a flowchart illustrating an example of a processing procedure and processing details of message classification model training processing in the processing procedure illustrated in FIG. 13.

FIG. 16 is a flowchart illustrating an example of an overall processing procedure and processing details of present bias analysis processing executed by the present bias analysis unit illustrated in FIG. 4 in a messaging phase.

FIG. 17 is a flowchart illustrating an example of an overall processing procedure and processing details of messaging control processing executed by the messaging control unit illustrated in FIG. 4 in a messaging phase.

FIG. 18 is a flowchart illustrating an example of a processing procedure and processing details of present bias estimation processing in the processing procedure illustrated in FIG. 17.

FIG. 19 is a flowchart illustrating an example of a processing procedure and processing details of message classification processing in the processing procedure illustrated in FIG. 17.

FIG. 20 is a flowchart illustrating an example of a processing procedure and processing details of message candidate extraction processing in the processing procedure illustrated in FIG. 17.

FIG. 21 is a flowchart illustrating an example of a processing procedure and processing details of message transmission adjustment processing in the processing procedure illustrated in FIG. 17.

FIG. 22 is a diagram illustrating an example of vital data stored in a vital data storage unit in the present bias analysis data storage unit illustrated in FIG. 4.

FIG. 23 is a diagram illustrating an example of action data stored in an action data storage unit in the present bias analysis data storage unit illustrated in FIG. 4.

FIG. 24 is a diagram illustrating an example of task data stored in a task data storage unit in the present bias analysis data storage unit illustrated in FIG. 4.

FIG. 25 is a diagram illustrating an example of preprocessed vital data.

FIG. 26 is a diagram illustrating an example of preprocessed action data.

FIG. 27 is a diagram illustrating an example of preprocessed task data.

FIG. 28 is a diagram illustrating an example of feature data after combining processing.

FIG. 29 is a diagram illustrating an example of feature data with time information.

FIG. 30 is a diagram illustrating an example of present bias data stored in a present bias data storage unit in the present bias analysis data storage unit illustrated in FIG. 4.

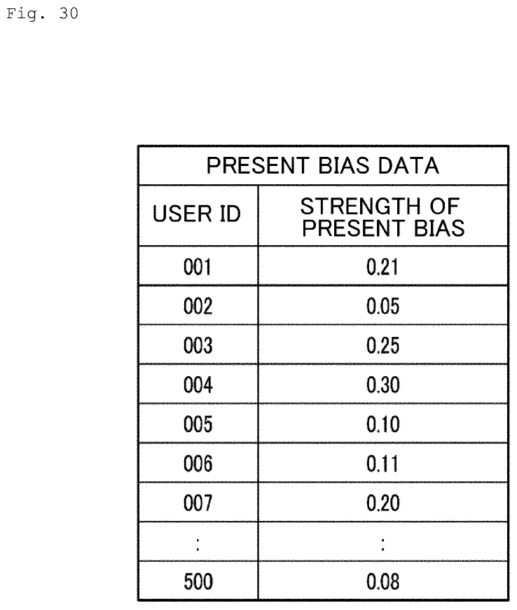

FIG. 31 is a diagram illustrating an example of a network structure of a present bias analysis model.

FIG. 32 is a diagram illustrating an example of parameters of the present bias analysis model.

FIG. 33 is a diagram illustrating an example of training messages stored in a training message data storage unit in the messaging control data storage unit illustrated in FIG. 5.

FIG. 34 is a diagram illustrating an example of token data stored in a token storage unit in the messaging control data storage unit illustrated in FIG. 5.

FIG. 35 is a diagram illustrating an example of data after message preprocessing.

FIG. 36 is a diagram illustrating an example of a training message label stored in a training message label storage unit in the messaging control data storage unit illustrated in FIG. 5.

FIG. 37 is a diagram illustrating an example of data after message classification processing.

FIG. 38 is a diagram illustrating an example of extracted message candidate data.

FIG. 39 is a diagram illustrating an example of information stored in a message transmission management information storage unit in the messaging control data storage unit illustrated in FIG. 5.

FIG. 40 is a diagram illustrating an example of a network structure of a message classification model.

FIG. 41 is a diagram illustrating an example of message classification model data stored in a message classification model storage unit in the messaging control data storage unit illustrated in FIG. 5.

### DESCRIPTION OF EMBODIMENTS

Embodiments according to this invention will be described below with reference to the drawings.

### One Embodiment

### Configuration Example

### (1) System

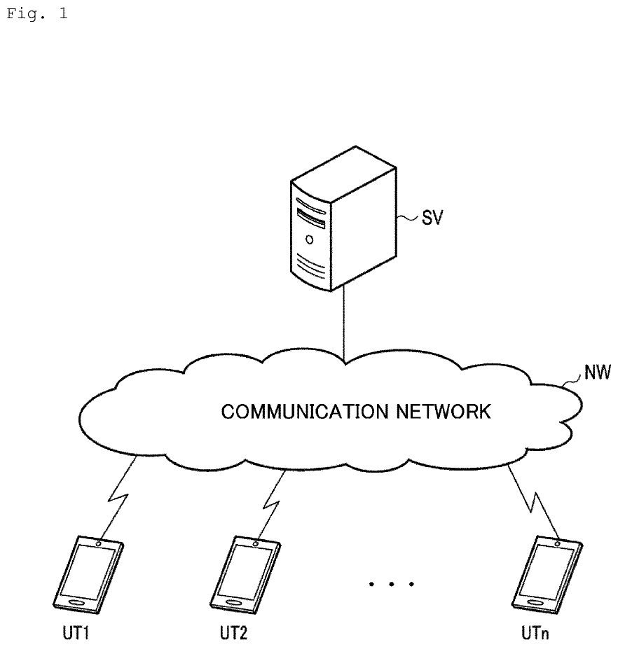

FIG. 1 is a diagram illustrating an example of a configuration of a life assistance system including a messaging device according to an embodiment of the present invention.

The system according to an embodiment enables data transmission via a network NW between a messaging device SV and user terminals UTI to UTn used by a plurality of users who are messaging targets.

As the user terminals UT1 to UTn, for example, a general-purpose smartphone including an input device, a display device, and a communication application such as a browser or a mailer is used.

The user terminals UT1 to UTn transmit a plurality of kinds of data necessary to estimate present biases of the users to the messaging device SV by executing an application/program installed in advance. More specifically, the user terminals UT1 to UTn transmit vital data measured by a health management application and action data and task data managed by a scheduler to the messaging device SV. In addition, the user terminals UT1 to UTn receive message data in which present biases have been reflected, which are transmitted from the messaging device SV.

As the user terminals UT1 to UTn, a wearable terminal, a tablet terminal, a personal computer, and the like may be used in addition to smartphones.

The network NW includes, for example, a wide area network with the Internet at the core thereof, and an access network for accessing the wide area network. As the access network, for example, a wired or wireless public communication network, a wired or wireless local area network (LAN), or a cable television (CATV) network is used, but the present invention is not limited thereto.

### (2) Messaging Device SV

### (2-1) Overall Configuration

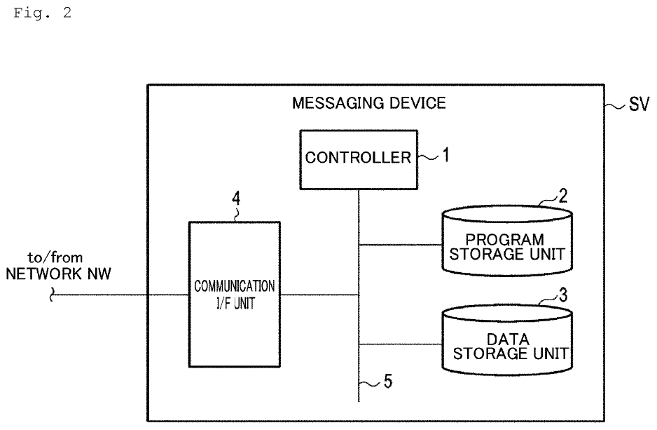

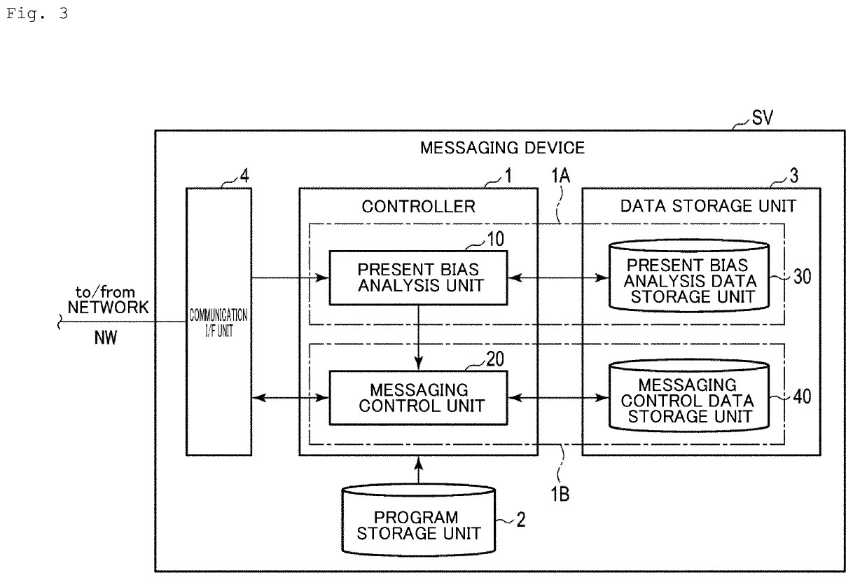

FIG. 2 and FIG. 3 are block diagrams respectively illustrating examples of a hardware configuration and a software configuration of the messaging device SV according to an embodiment of the present invention.

The messaging device SV is configured by, for example, a server computer disposed on a Web or a cloud. The messaging device SV includes a controller 1 using a hardware processor such as a central processing unit (CPU), and a storage unit including a program storage unit 2 and a data storage unit 3 and a communication interface (hereinafter, the interface is referred to as I/F) unit 4 are connected to the controller 1 via a bus 5.

The communication I/F unit 4 performs access control for the network NW and transmission and reception of data under the control of the controller 1. More specifically, the communication I/F unit 4 is used to receive vital data, action data, and task data of users, and data indicating the kind of a target action transmitted from the user terminals UT1 to UTn and to transmit message data reflecting estimation results of present biases of the users generated by the controller 1 to the user terminals UT1 to UTn. The communication I/F unit 4 is also used, for example, at the time of acquiring message data from a manager terminal of the system.

The program storage unit 2 is, for example, a combination of a non-volatile memory such as a hard disk drive (HDD) or a solid state drive (SSD) capable of writing and reading at any time and a non-volatile memory such as a read only memory (ROM) as a storage medium, and stores various programs necessary for execution of various types of control processing according to an embodiment of the present invention in addition to middleware such as an operating system (OS).

The data storage unit 3 is, for example, a combination of a non-volatile memory such as an HDD or an SSD capable of writing and reading at any time, and a volatile memory such as a random access memory (RAM) as a storage medium, and in the storage area thereof, a present bias analysis data storage unit 30 and a messaging control data storage unit 40 are provided as storage units according to an embodiment.

The controller 1 includes a present bias analysis unit 10 and a messaging control unit 20 as processing functions for executing an embodiment of the present invention. All of the units 10 and 20 are realized by a hardware processor of the controller 1 executing an application/program stored in the program storage unit 2.

Some or all of the processing units included in the aforementioned present bias analysis unit 10 and messaging control unit 20 may be realized using hardware such as a large scale integration (LSI) or an application specific integrated circuit (ASIC).

In the messaging device SV of an embodiment, a present bias analyzer 1A is configured by the present bias analysis unit 10 and the present bias analysis data storage unit 30, and a messaging controller 1B is configured by the messaging control unit 20 and the messaging control data storage unit 40.

### (2-2) Present Bias Analyzer 1A

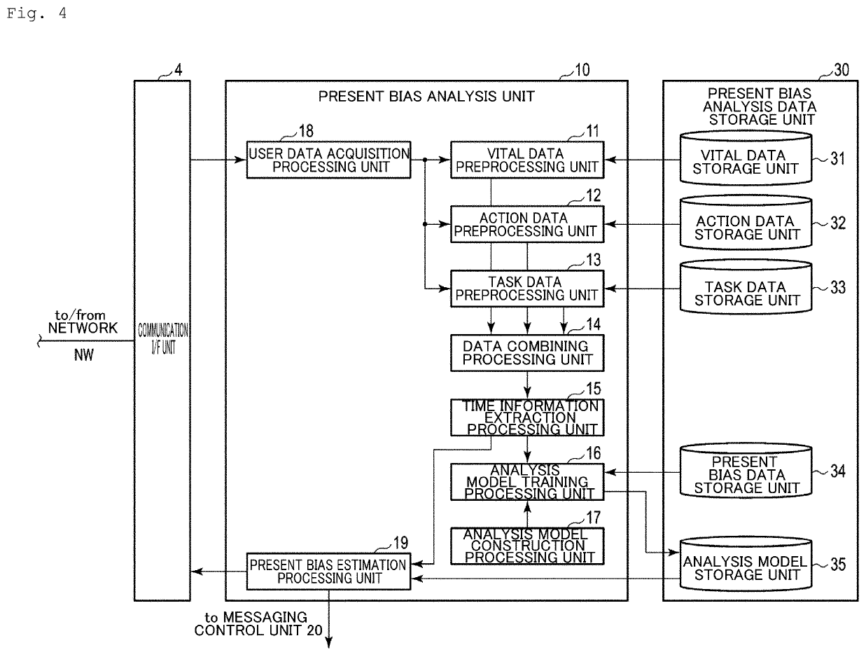

FIG. 4 is a block diagram illustrating an example of a configuration of the present bias analyzer 1A.

The present bias analysis data storage unit 30 includes a vital data storage unit 31, an action data storage unit 32, a task data storage unit 33, a present bias data storage unit 34, and an analysis model storage unit 35 provided therein.

The vital data storage unit 31 stores a plurality of pieces of vital data measured in a certain period as data for present bias learning with respect to a plurality of users. Similarly, the action data storage unit 32 and the task data storage unit 33 store data representing actions and tasks executed by the plurality of users in the certain period as data for present bias learning.

The present bias data storage unit 34 stores known data of present biases of the plurality of users that are learning targets as training data for present bias learning.

The analysis model storage unit 35 is used to store data representing a trained present bias analysis model.

The present bias analysis unit 10 includes, as processing functions necessary to estimate a present bias of a user, a vital data preprocessing unit 11, an action data preprocessing unit 12, a task data preprocessing unit 13, a data combining processing unit 14, a time information extraction processing unit 15, an analysis model training processing unit 16, an analysis model construction processing unit 17, a user data acquisition processing unit 18, and a present bias estimation processing unit 19.

The vital data preprocessing unit 11 reads vital data of a learning target from the vital data storage unit 31 in a present bias learning phase, and receives vital data series of estimation target users transmitted from the user terminals UT1 to UTn in a messaging phase. Then, the vital data preprocessing unit 11 calculates summary statistics for each time interval with a preset duration for the acquired vital data, and outputs data including the calculation result as preprocessed vital data.

The action data preprocessing unit 12 reads action data of a learning target from the action data storage unit 32 in the present bias learning phase, and receives action data of target users transmitted from the user terminals UT1 to UTn in the messaging phase. Then, the action data preprocessing unit 12 performs data aggregation by deleting, for example, the same data or a small amount of data for the acquired action data, and assigns, to aggregated actions, action IDs indicating the types of the actions and time information. Then, the generated data is output as preprocessed action data.

The task data preprocessing unit 13 reads task data of a learning target from the task data storage unit 33 in the present bias learning phase, and receives task data of estimation target users transmitted from the user terminals UT1 to UTn in the messaging phase. Then, the task data preprocessing unit 13 calculates, for example, a goal achievement rate and a goal progress rate for the received task data, aggregates the calculation results for each time interval, and outputs the data obtained thereby as preprocessed task data.

The data combining processing unit 14 combines the preprocessed vital data, action data, and task data, which have the same time intervals, for each user, thereby generating combined feature data for each user.

The time information extraction processing unit 15 extracts time information represented by “month”, “day of the week”, “date”, and “time” for each time interval from the combined feature data of each user, and generates feature data with time information by adding the extracted time information to the combined feature data.

The analysis model construction processing unit 17 constructs a present bias analysis model for handling the feature data with time information using a network structure of a deep neural network (DNN).

The analysis model training processing unit 16 learns each parameter of the present bias analysis model constructed by the analysis model construction processing unit 17 using feature data with time information of each user that is a learning target and present bias known data of each user that is a learning target stored in the present bias data storage unit 34 in the present bias learning phase. Then, the trained present bias analysis model is stored in the analysis model storage unit 35.

The user data acquisition processing unit 18 acquires a vital data series, an action data series and a task data series of a user that is a messaging target from the user terminals UT1 to UTn used by the estimation target users through the communication I/F unit 4 in the messaging phase.

The present bias estimation processing unit 19 inputs feature data with time information of the user that is a messaging target generated by the time information extraction processing unit 15 to the trained present bias analysis model stored in the analysis model storage unit 35 in the messaging phase. Then, estimation data of a present bias output from the trained present bias analysis model is output to the messaging control unit 20.

### (2-3) Messaging Controller 1B

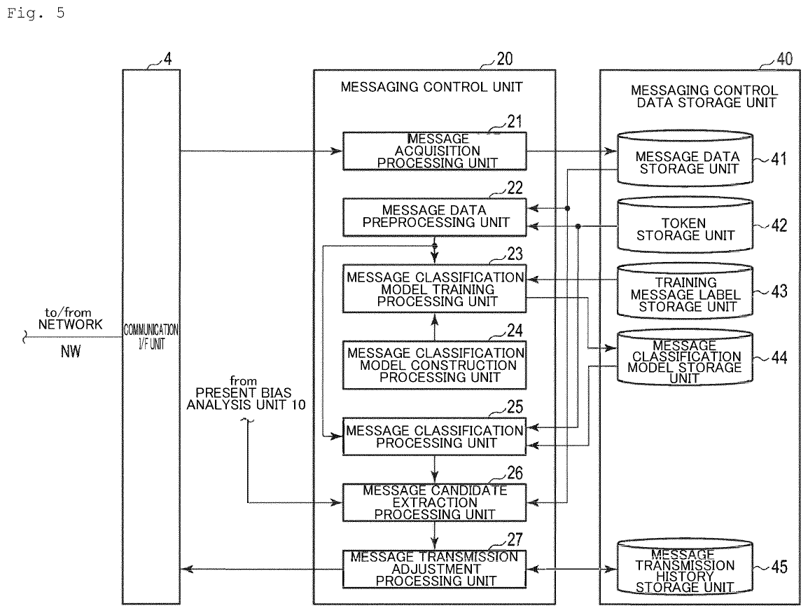

FIG. 5 is a block diagram illustrating an example of a configuration of the messaging controller 1B.

The messaging control data storage unit 40 includes a message data storage unit 41, a token storage unit 42, a training message label storage unit 43, a message classification model storage unit 44, and a message transmission history storage unit 45 provided therein.

A plurality of pieces of training message data used for model training in a message learning phase and a message list used in a messaging phase are stored in the message data storage unit 41 in association with message identification information (hereinafter, identification information is referred to as an ID). Message data is represented by, for example, text data.

The token storage unit 42 stores a plurality of words (tokens) constituting message data as a token dictionary in a state of being associated with token IDs.

In the training message label storage unit 43, data representing a reward type and a target action as a training message label is stored in association with a message ID for each piece of message data.

The message classification model storage unit 44 is used to store data representing a trained message classification model.

In the message transmission history storage unit 45, transmission history data indicating the number of times of transmission of a message and a final transmission time is stored in association with a user ID of a transmission destination and a message ID.

The messaging control unit 20 includes, as processing functions necessary to perform messaging control, a message acquisition processing unit 21, a message data preprocessing unit 22, a message classification model training processing unit 23, a message classification model construction processing unit 24, a message classification processing unit 25, a message candidate extraction processing unit 26, and a message transmission adjustment processing unit 27.

The message acquisition processing unit 21 acquires a plurality of pieces of training message data used for message classification model training in a message learning phase and a message list used in a messaging phase from a system manager terminal or the like, for example, and stores each piece of the acquired message data in the message data storage unit 41 in association with a message ID. The message data storage unit 41 may be divided into a storage unit for storing training message data and a storage unit for storing a message list.

The message data preprocessing unit 22 reads message data from the message data storage unit 41 and decomposes the read message data into words. Then, the message data preprocessing unit 22 extracts token IDs corresponding to the words with reference to a token dictionary stored in the token storage unit 42, and creates a token list of the message. An example of this message data preprocessing will be described in an operation example.

The message classification model construction processing unit 24 creates data representing a network structure of a message classification model. The network structure of the message classification model is also configured by, for example, a DNN.

The message classification model training processing unit 23 reads preprocessed message data from the message data preprocessing unit 22 and message label data from the training message label storage unit 43, and learns parameters of the message classification model created by the message classification model construction processing unit 24 on the basis of the read data. Then, data representing the learned parameters and the model structure are stored in the message classification model storage unit 44 as data representing the trained message classification model. An example of this message classification model training processing will be described in an operation example.

The message classification processing unit 25 inputs the preprocessed message data output from the message data preprocessing unit 22 to the trained message classification model stored in the message classification model storage unit 44 to acquire data representing a reward type of the message data and a type of a target action from the trained message classification model. Then, the acquired data representing the reward type and the type of the target action is output as classified message data.

The message candidate extraction processing unit 26 extracts message IDs corresponding to preset conditions from the classified message data output from the message classification processing unit 25 on the basis of present bias estimation data output from the present bias analysis unit 10 and data representing target actions input from the user terminals UT1 to UTn. Then, the message candidate extraction processing unit 26 reads relevant message data from the message data storage unit 41 on the basis of the message IDs, generates a data table having the read message data, message IDs thereof, a reward type, and a target action as attributes, and outputs the generated data table as message data candidates.

The message transmission adjustment processing unit 27 retrieves the relevant number of times of transmission or the latest transmission time from the message transmission history storage unit 45 on the basis of the message IDs included in the message data candidates and user IDs. Then, the message transmission adjustment processing unit 27 selects one message ID for which the number of times of transmission or the latest transmission time satisfies preset transmission conditions, and transmits message data corresponding to the selected message ID to the user terminal UT1 to UTn of a target user through the communication I/F unit 4. In addition, the message transmission adjustment processing unit 27 updates the corresponding number of times of transmission and the latest transmission time stored in the message transmission history storage unit 45 after transmission of the message data.

### Operation Examples

Next, operation examples of the messaging device SV configured as described above will be described.

### (1) Present Bias Learning Phase

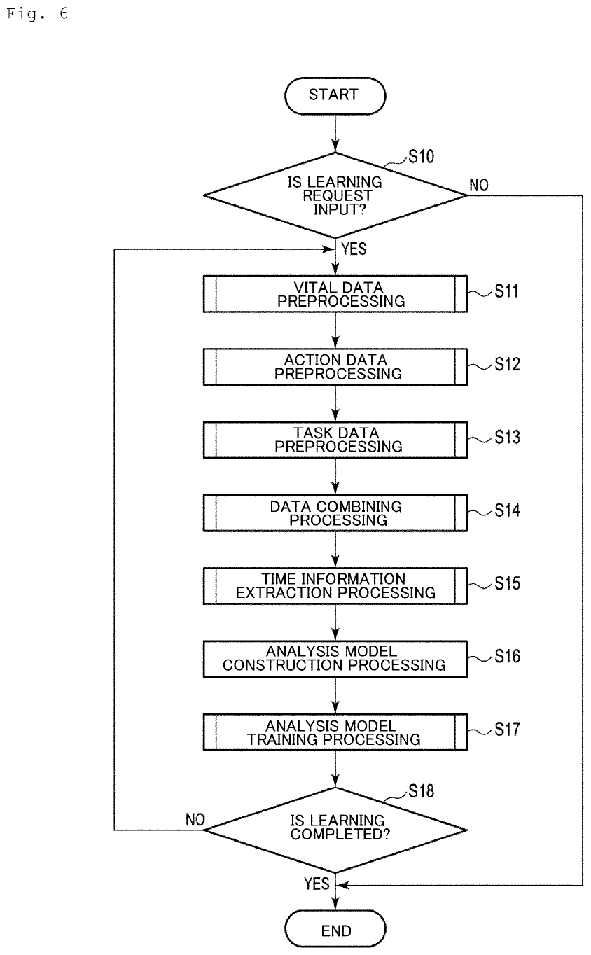

FIG. 6 is a flowchart illustrating an example of a processing procedure and processing details of learning processing executed by the present bias analysis unit 10 of the messaging device SV in a present bias learning phase.

It is assumed that vital data, action data, and task data of a plurality of users that are learning targets are stored in advance in the vital data storage unit 31, the action data storage unit 32, and the task data storage unit 33. Further, it is assumed that known data of present biases of the plurality of users that are learning targets are stored in advance in the present bias data storage unit 34.

In this state, for example, when a system manager inputs a present bias learning request through a manager terminal that is not illustrated and the present bias analysis unit 10 of the messaging device SV detects the learning request in step S10, the present bias analysis unit 10 of the messaging device SV sets a present bias learning phase and then executes present bias analysis model training processing as follows.

### (1-1) Preprocessing of Vital Data

The present bias analysis unit 10 of the messaging device SV first performs preprocessing of vital data under the control of the vital data preprocessing unit 11 in step S11 as follows.

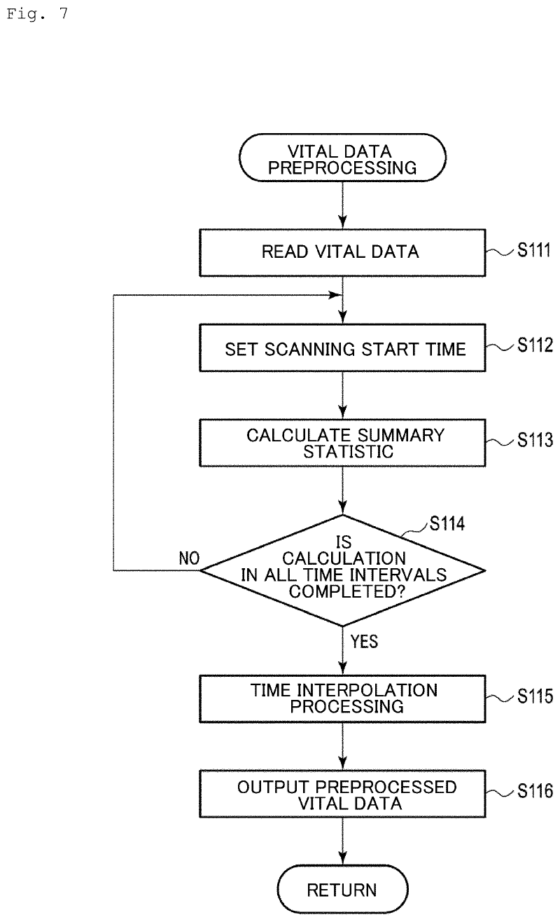

FIG. 7 is a flowchart illustrating an example of a processing procedure and processing details of preprocessing of vital data executed by the vital data preprocessing unit 11.

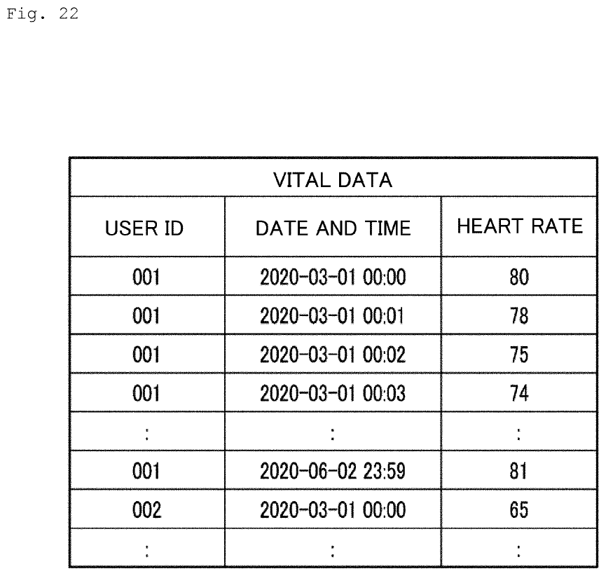

That is, the vital data preprocessing unit 11 first reads vital data from the vital data storage unit 31 in step S111. Vital data is obtained by associating a measured heart rate and measurement date and time with identification information of a user (hereinafter referred to as a user ID) for each entry of each row in FIG. 22, for example.

The vital data preprocessing unit 11 sets a start time of each time interval with a preset fixed duration for the read vital data in step S112, and calculates summary statistics of heart rates measured in each time interval in step S113. As summary statistics, for example, the mean, standard deviation, maximum and minimum values of heart rates are calculated.

The vital data preprocessing unit 11 determines whether or not calculation for all time intervals of the vital data ends in step S114 every time processing of calculating the summary statistics for one time interval ends. If time intervals for which calculation is not performed remains, processing returns to step S112 to set the next time interval, and the summary statistic calculation processing is executed in step S113. Thereafter, summary statistic calculation processing for each time interval for which calculation is not performed is repeated.

On the other hand, when calculation for all time intervals ends, the vital data preprocessing unit 11 proceeds to step S115 and performs interpolation processing on data between time intervals in this step. Then, the vital data including the data after interpolation processing is output to the data combining processing unit 14 as preprocessed vital data VD1 in step S116.

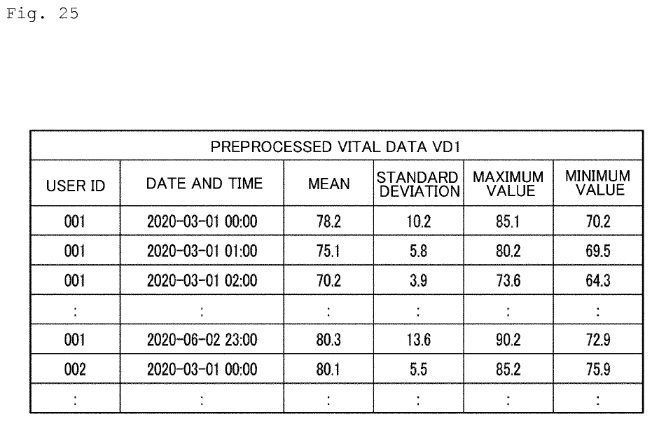

FIG. 25 is a diagram illustrating an example of the preprocessed vital data VD1.

### (1-2) Preprocessing of Action Data

Next, the present bias analysis unit 10 of the messaging device SV performs preprocessing of action data under the control of the action data preprocessing unit 12 in step S12 as follows.

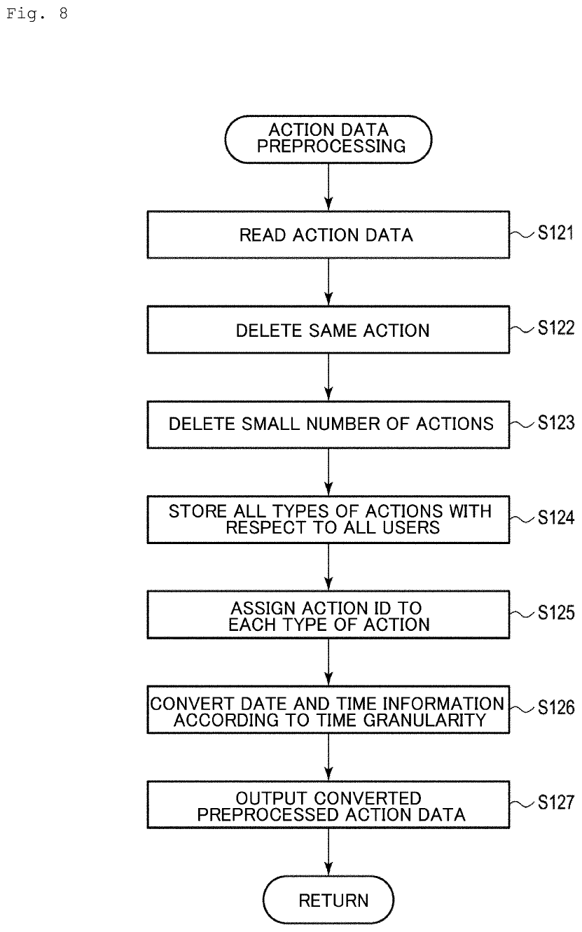

FIG. 8 is a flowchart illustrating an example of a processing procedure and processing details of action data preprocessing executed by the action data preprocessing unit 12.

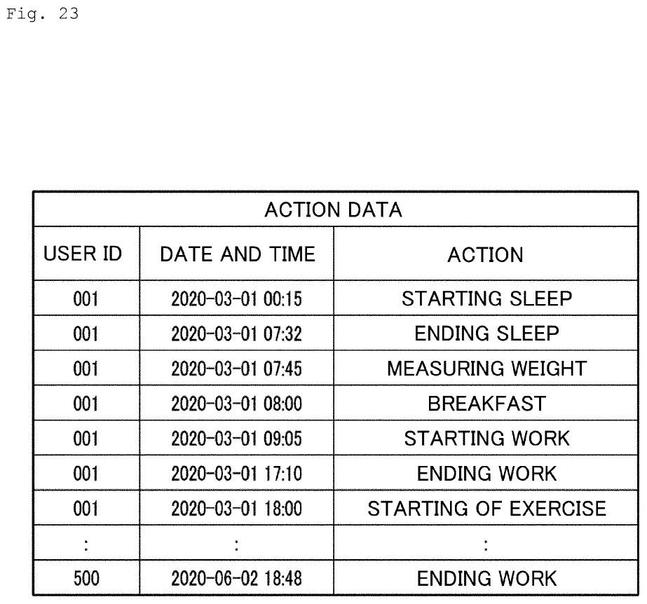

That is, the action data preprocessing unit 12 first reads action data of each user that is a learning target from the action data storage unit 32 in step S121. Action data is obtained by associating the content of an action and execution date and time with a user ID for each entry shown as a row in FIG. 23, for example.

The action data preprocessing unit 12 first sequentially compares entries in step S122 for each user with respect to the read action data of the learning target. If the same action continuously appears within a preset short time, for example, one of the actions is left and the other entries are deleted. For example, when an action of “starting exercise” of a certain user appears a predetermined number of times or more in a period with a predetermined time span, only “starting start” appearing first is left, and the other same “starting exercise” is regarded as being erroneously registered and deleted. The duration of the time span may be arbitrarily set by the system manager.

Subsequently, the action data preprocessing unit 12 compares the actions of respective entries and deletes the entry of an action whose number of observations does not reach a fixed number in step S123. For example, the number of times of appearance is counted for each type of action, and an action whose number of times of appearance does not reach a predetermined number of times set by the system manager is deleted. By performing each type of deletion processing, the entries can be aggregated for action data.

The action data preprocessing unit 12 executes aggregation of the entries in steps S122 and S123 for the entries of all users. Then, entries of actions of all users remaining as a result of aggregation of the entries are temporarily stored, and action IDs for identifying the kinds of the stored actions are assigned to the stored actions in step S125.

Finally, the action data preprocessing unit 12 converts the date and time included in each entry of the action data into time information according to the granularity of the time interval defined in the vital data preprocessing in step S126. Then, the action data having the converted time information is output to the data combining processing unit 14 as preprocessed action data AD1 in step S127.

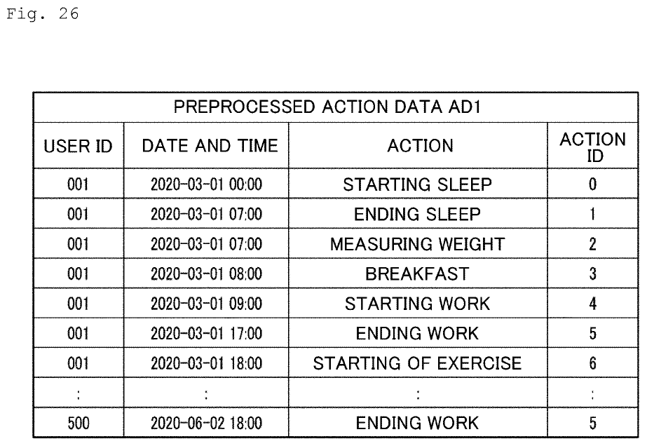

FIG. 26 is a diagram illustrating an example of the preprocessed action data AD1.

### (1-3) Preprocessing of Task Data

Subsequently, the present bias analysis unit 10 of the messaging device SV performs task data preprocessing under the control of the task data preprocessing unit 13 in step S13 as follows.

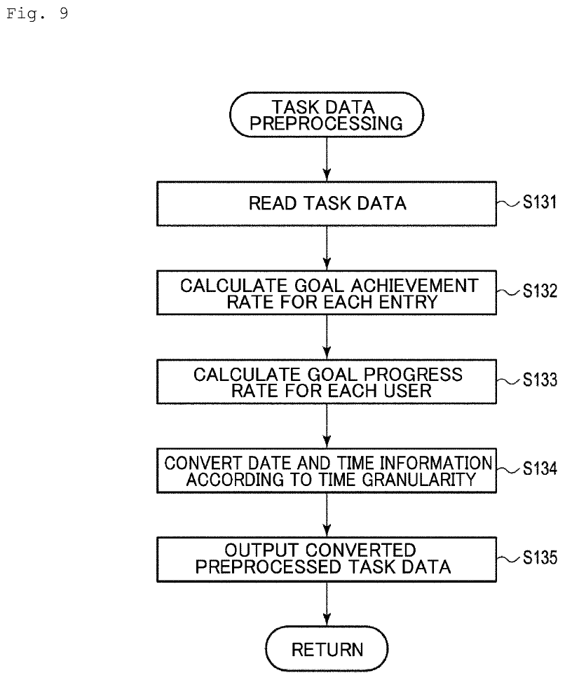

FIG. 9 is a flowchart illustrating an example of a processing procedure and processing details of task data preprocessing executed by the task data preprocessing unit 13.

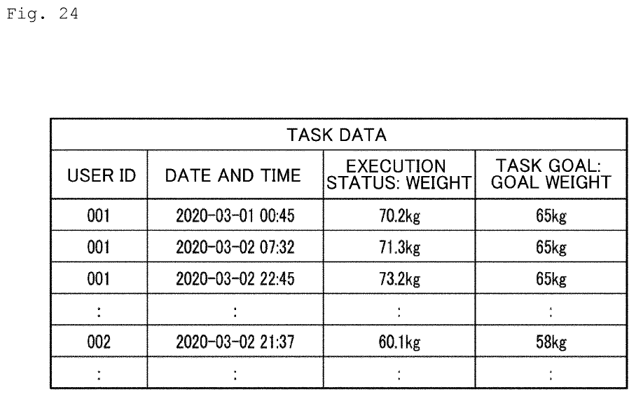

That is, the task data preprocessing unit 13 first reads task data from the task data storage unit 33 in step S131. Task data is composed of a user ID, and the content of a task and the execution date and time of the task associated with the user ID for each entry shown as a row in FIG. 24, for example. In this example, the contents of tasks are related to diet and are represented by present weights representing execution situations and target weights.

The task data preprocessing unit 13 first calculates a goal achievement rate for each entry in step S132 for the read task data. Then, a goal progress rate for each user is calculated in step S133. Subsequently, the task data preprocessing unit 13 converts the date and time included in each entry of the task data into time information according to the granularity of the time interval defined in preprocessing of vital data in advance in step S134. Then, the task data having converted time information is output to the data combining processing unit 14 as preprocessed task data TD1 in step S135.

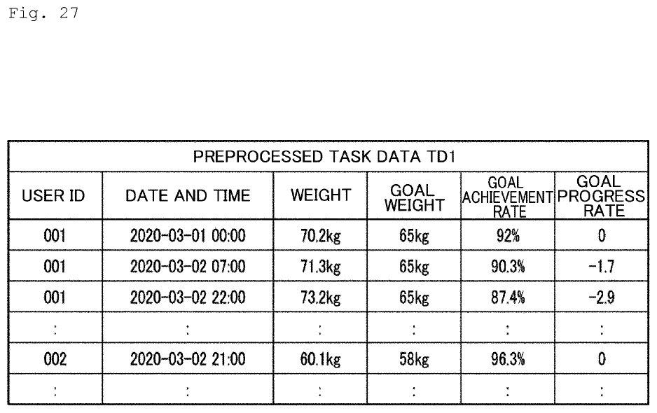

FIG. 27 is a diagram illustrating an example of the preprocessed task data TD1.

### (1-4) Data Combining Processing

Next, the present bias analysis unit 10 of the messaging device SV performs data combining processing under the control of the data combining processing unit 14 in step S14 as follows.

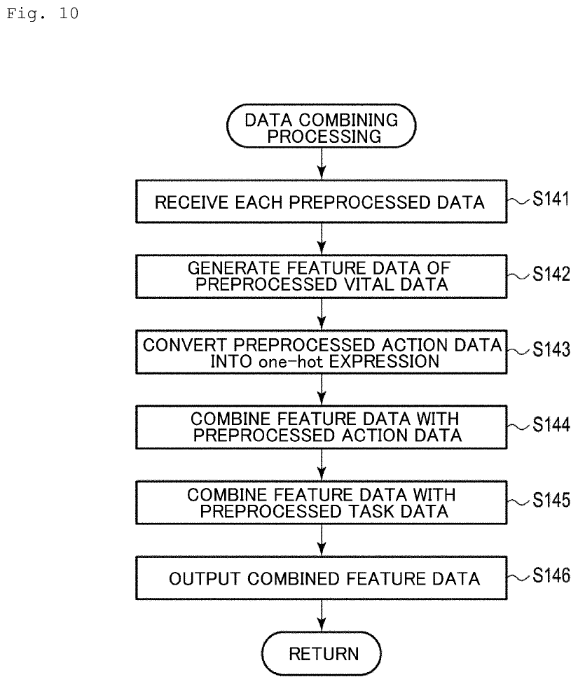

FIG. 10 is a flowchart illustrating an example of a processing procedure and processing details of data combining processing executed by the data combining processing unit 14.

That is, the data combining processing unit 14 first receives the preprocessed vital data VD1, action data AD1, and task data TD1 from the vital data preprocessing unit 11, the action data preprocessing unit 12, and the task data preprocessing unit 13 in step S141. Then, the data combining processing unit 14 generates a copy of the vital data VD1 after preprocessing, and defines the copy as feature data VD2 of the vital data in step S142.

Next, the data combining processing unit 14 converts the action ID of the action data AD1 after preprocessing into a one-hot expression in step S143. For example, a vector having the same dimension as the number of action types is prepared, only the dimension of a numerical value representing the action ID is converted to “1”, and the other dimensions are converted to “0”. Then, the data combining processing unit 14 combines the preprocessed action data AD2 in which the expression of the action ID has been converted, with the feature data of the vital data by the same users and by matching time intervals on the basis of the user IDs and time information in step S144.

Further, the data combining processing unit 14 combines the preprocessed task data TD2 with the feature data of the vital data by the same users and by matching the time intervals on the basis of the user IDs and the time information in step S145. Then, the data combining processing unit 14 outputs the combined feature data to the time information extraction processing unit 15 in step S146.

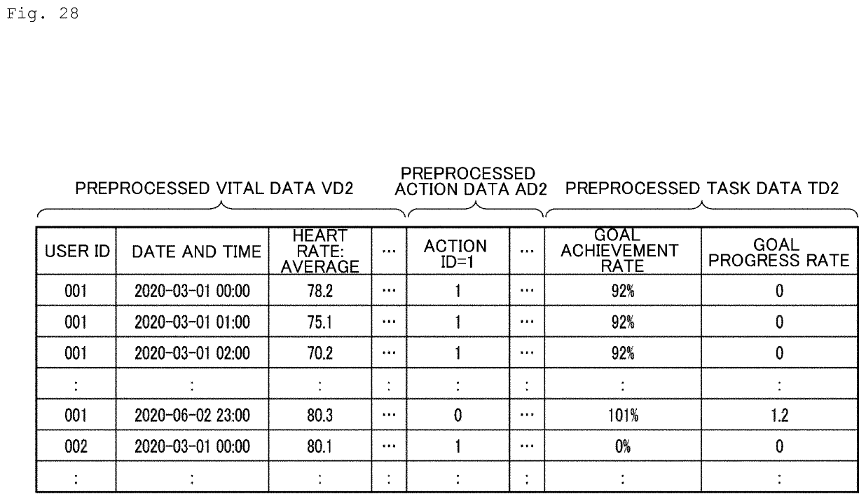

FIG. 28 illustrates an example of the combined feature data, where VD2 indicates preprocessed vital data, AD2 indicates preprocessed action data, and TD2 indicates preprocessed task data.

### (1-5) Extraction of Time Information

Next, the present bias analysis unit 10 of the messaging device SV performs time information extraction processing under the control of the time information extraction processing unit 15 in step S15 as follows.

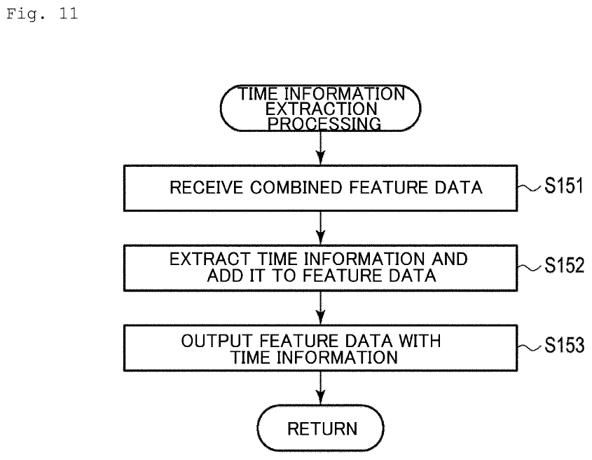

FIG. 11 is a flowchart illustrating an example of a processing procedure and processing details of time information extraction processing executed by the time information extraction processing unit 15.

That is, the time information extraction processing unit 15 first receives combined feature data from the data combining processing unit 14 in step S151. Then, the time information extraction processing unit 15 extracts, for each entry of the combined feature data, “month”, “data of the week”, “date”, and “time” constituting the time information on the basis of the date and time included in the entry, and adds the elements of the extracted time information to the entries of the combined feature data in step S152.

Finally, the time information extraction processing unit 15 outputs the combined feature data (hereinafter referred to as feature data with time information) after addition of the time information to the analysis model training processing unit 16 in step S153.

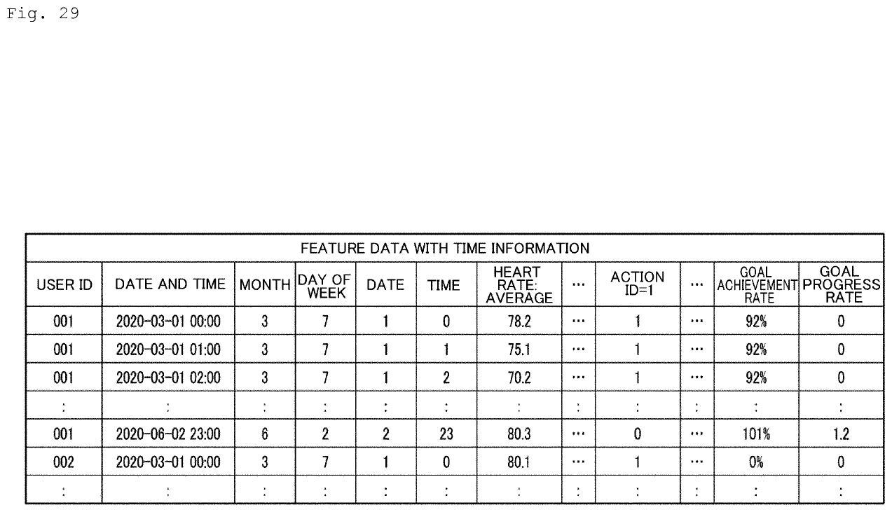

FIG. 29 illustrates an example of feature data with time information output from the time information extraction processing unit 15.

### (1-6) Construction of Present Bias Analysis Model

Next, the present bias analysis unit 10 of the messaging device SV constructs a present bias analysis model through the analysis model construction processing unit 17 in step S16. The present bias analysis model is configured using, for example, a DNN, receives feature data for each user as inputs, and outputs an estimation value of the strength of a present bias of each user.

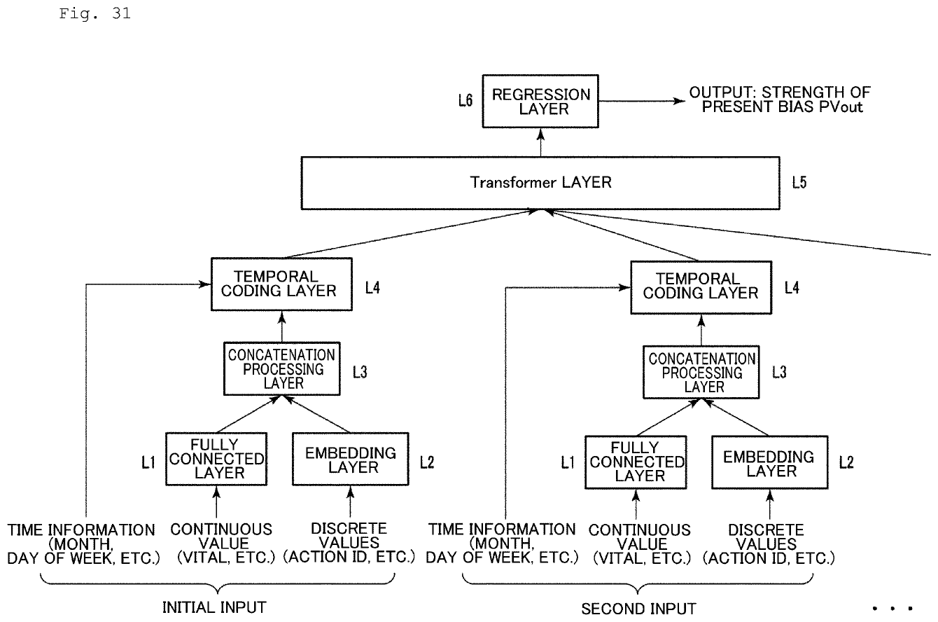

FIG. 31 illustrates an example of a network structure of the present bias analysis model constructed by the analysis model construction processing unit 17. That is, the present bias analysis model has a configuration in which a fully connected layer L1 and an embedding layer L2 are arranged as input layers, and a concatenation processing layer L3, a temporal coding layer L4, a transformer layer L5, and a regression layer L6 are arranged in order.

The fully connected layer L1 extracts abstract features from continuous values of input feature data. More specifically, an embedding layer having the number of data strings of continuous values as an input is prepared, converted into a feature vector of a specific number of dimensions, and output.

The embedding layer L2 extracts abstract features from category values in the input feature data. For example, an embedding layer having the number of dimensions of the number of types of actions as an input is prepared, converted into a feature vector of a specific number of dimensions, and output.

The concatenation processing layer L3 concatenates the two feature vectors output from the fully connected layer L1 and the embedding layer L2. For example, the two feature vectors are concatenated in the lateral direction, and the concatenated feature vectors are output.

The temporal coding layer L4 assigns time information to the feature vector. For example, time information in feature data with the time information is input to the fully connected layer and then nonlinearly transformed using an activation function (for example, a Sigmoid function, a ReLU function, or the like), and the converted feature vector is output.

The transformer layer L5 further abstracts the abstracted feature vector as series data. Specifically, the series data is received sequentially and repeatedly nonlinearly transformed.

The regression layer L6 converts the feature vector transformed by the transformer layer into a scalar value PVout corresponding to the strength of the present bias and outputs the scalar value.

### (1-7) Training of Present Bias Analysis Model

Next, the present bias analysis unit 10 of the messaging device SV executes processing of training the present bias analysis model constructed by the analysis model construction processing unit 17 in step S17 under the control of the analysis model training processing unit 16 as follows.

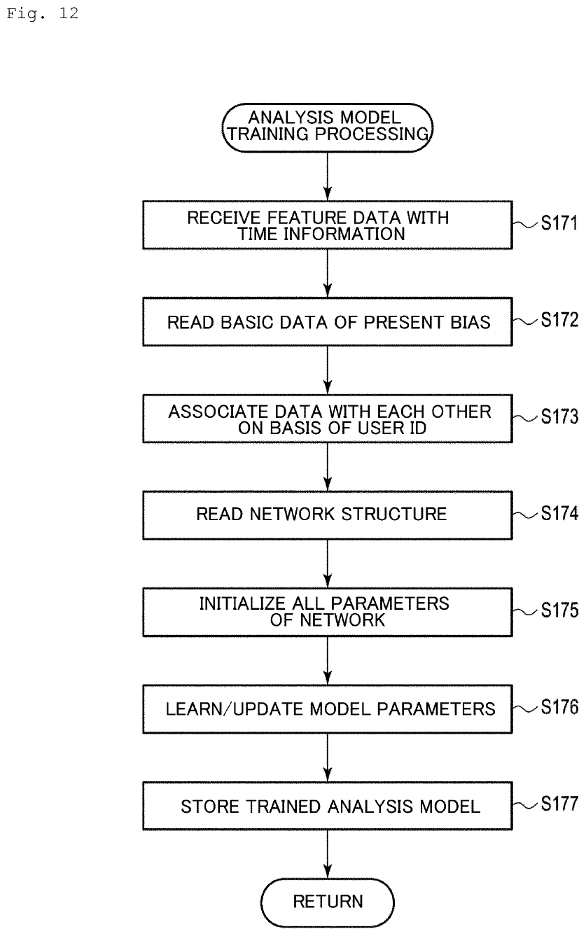

FIG. 12 is a flowchart illustrating an example of a processing procedure and processing details of present bias analysis model training processing executed by the analysis model training processing unit 16.

That is, the analysis model training processing unit 16 first receives feature data with time information that is a learning target from the time information extraction processing unit 15 in step S171. Further, the analysis model training processing unit 16 reads known data of the present bias of each user that is a learning target from the present bias data storage unit 34 in step S172. Then, the analysis model training processing unit 16 associates each entry of the feature data with time information with the known data of the present bias on the basis of user IDs in step S173.

Next, the analysis model training processing unit 16 receives the network structure of the present bias analysis model from the analysis model construction processing unit 17 in step S174. Then, the analysis model training processing unit 16 initializes parameters of the respective layers LI to L6 of the received network structure in step S175. Specifically, each parameter is initialized using a normally distributed random number with a mean of “0” and a standard deviation of “1”.

Next, the analysis model training processing unit 16 learns each parameter of the network structure using the input feature data with time information and the known data of the present bias for each user ID in step S176. Specifically, each time an entry of the feature data with time information is input, each parameter of the network structure is learned and updated using preprocessed vital data VD2, action data AD2, and task data TD2 included in the entry and known data of the present bias of the learning target user corresponding thereto.

Then, when learning/updating processing using all entries of the input feature data with time information ends, the analysis model training processing unit 16 stores the network structure and each parameter of the network structure for which the learning/updating processing ends in the analysis model storage unit 35 as a trained present bias analysis model MD in step S177.

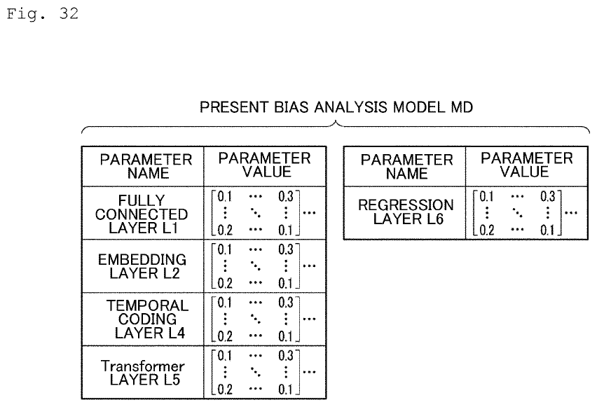

FIG. 32 illustrates an example of the trained present bias analysis model MD stored in the analysis model storage unit 35.

### (2) Message Learning Phase

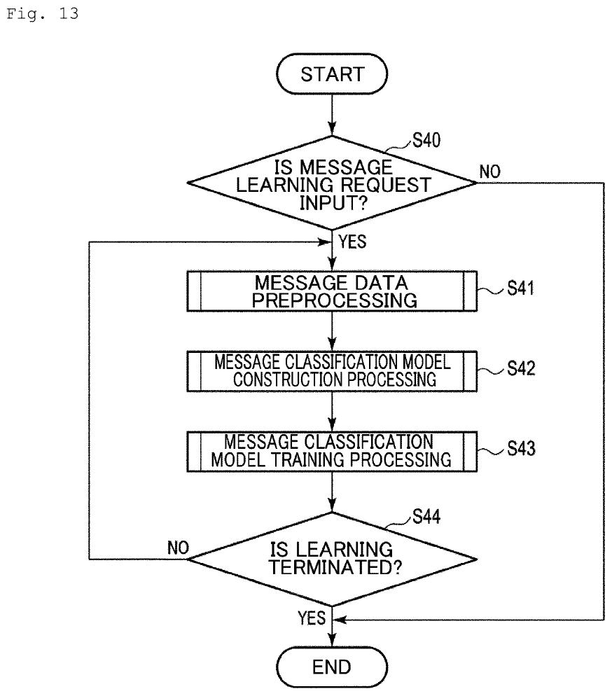

FIG. 13 is a flowchart illustrating an example of a processing procedure and processing details of message classification model training processing executed by the messaging control unit 20 of the messaging device SV in a message learning phase.

It is assumed that a plurality of pieces of training message data, a token dictionary, and label data are stored in advance in the message data storage unit 41, the token storage unit 42, and the training message label storage unit 43.

In this state, for example, when the system manager inputs a message learning request through a manager terminal that is not illustrated, and the messaging control unit 20 of the messaging device SV detects the learning request in step S40, the messaging control unit 20 of the messaging device SV sets a message learning phase, and then executes message classification model training processing as follows.

### (2-1) Preprocessing of Message Data

The messaging control unit 20 of the messaging device SV first performs message data preprocessing under the control of the message data preprocessing unit 22 in step S41 as follows.

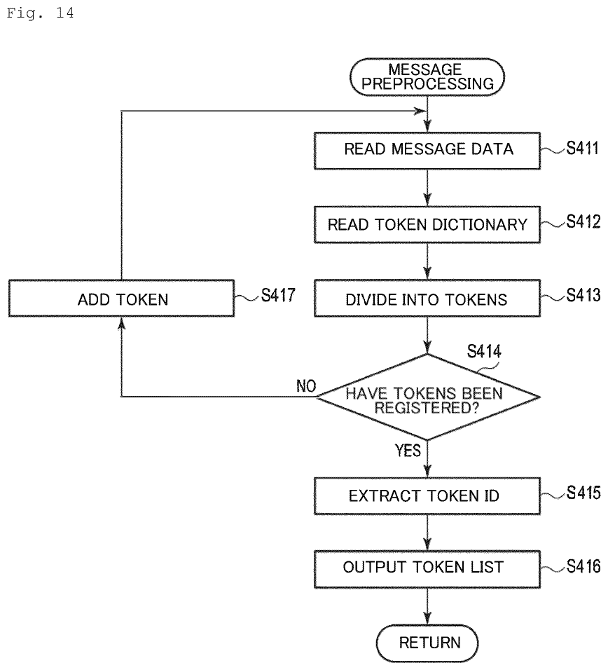

FIG. 14 is a flowchart illustrating an example of a processing procedure and processing details of message data preprocessing executed by the message data preprocessing unit 22.

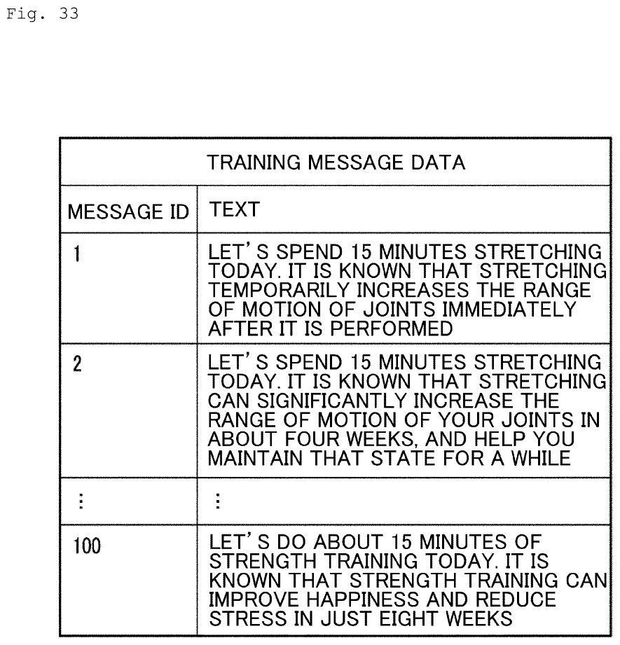

That is, the message data preprocessing unit 22 first reads one piece of message data from the message data storage unit 41 in step S411. As illustrated in FIG. 33, for example, text data representing a message is associated with a message ID in message data.

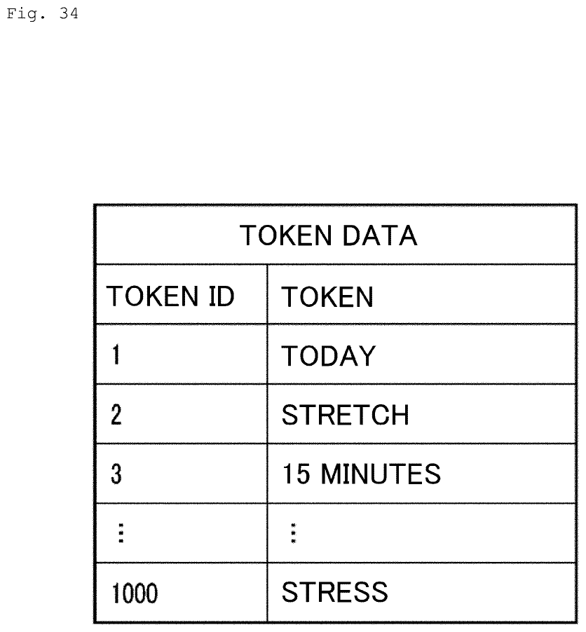

Subsequently, the message data preprocessing unit 22 reads the token dictionary from the token storage unit 42 in step S412. As illustrated in FIG. 34, for example, each token is registered in the token dictionary in association with a token ID.

Next, the message data preprocessing unit 22 divides the read message data into a plurality of words according to, for example, morphological analysis in step S413, and determines whether or not each divided word is included in the token dictionary in step S414. If a word is included in the token dictionary as a result of the determination, a token ID corresponding to the word is extracted from the token dictionary in step S145. The message data preprocessing unit 22 finally generates a token list composed of the extracted token ID in step S146, and outputs the generated token list as preprocessed message data.

Thereafter, the message data preprocessing unit 22 generates a token list by performing preprocessing on message data in steps S412 to S417 every time one piece of message data is read from the message data storage unit 41 in the same manner, and outputs the generated token list as preprocessed message data.

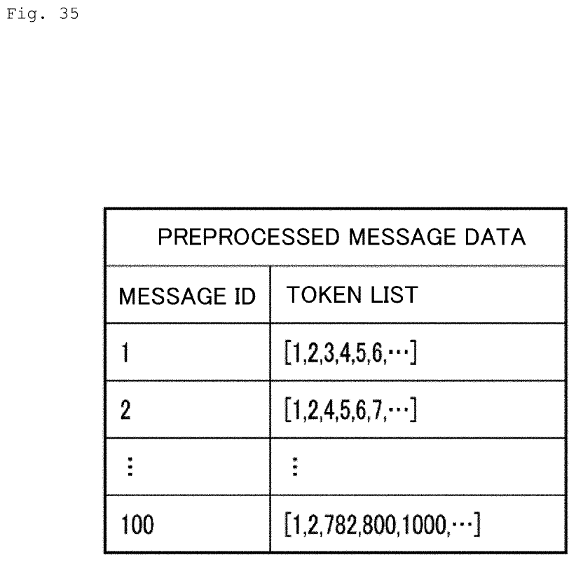

FIG. 35 illustrates an example of preprocessed message data.

If a word is not included in the token dictionary, the message data preprocessing unit 22 issues a numerical value obtained by adding 1 to the number of tokens in the token dictionary as a new token ID in step S417, and additionally registers the word in the token dictionary in association with the token ID.

### (2-2) Construction of Message Classification Model

Next, the messaging control unit 20 of the messaging device SV constructs a message classification model through the message classification model construction processing unit 24 in step S42. The message classification model is configured using, for example, a DNN, receives token data of a message as inputs, and outputs data representing a reward type and a target action of the message.

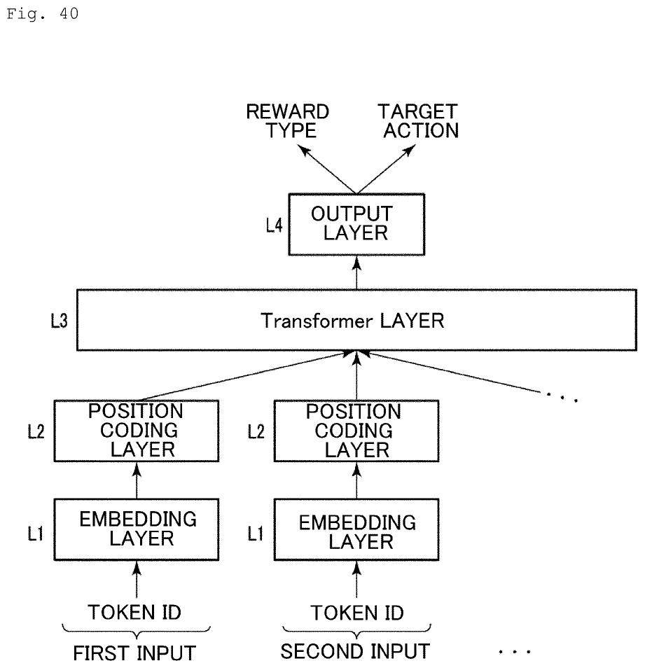

FIG. 40 illustrates an example of a network structure of the message classification model constructed by the message classification model construction processing unit 24. That is, the message classification model has a configuration in which an embedding layer L1 is arranged as an input layer, and a position coding layer L2, a transformer layer L3, and an output layer L4 are arranged in order.

The embedding layer L1 extracts an abstract feature from an input token ID. For example, an embedding layer having the number of dimensions of the number of token types as an input is prepared, converted into a feature vector of a specific number of dimensions, and output.

The position coding layer L2 embeds position information of a token. More specifically, the position information of the token is converted by a trigonometric function having a specific frequency and added to a feature vector obtained in the preceding stage layer. Time information is assigned to the feature vector.

The transformer layer L3 further abstracts the abstracted feature vector as series data. Specifically, the series data is received sequentially and repeatedly nonlinearly transformed.

The output layer L4 obtains a desired class label from the feature vector transformed by the transformer layer. For example, a vector for classifying reward types of messages and a vector for classifying target actions are output, and a class label corresponding to an index of a vector having the maximum value is output.

### (2-3) Training of Message Classification Model

Next, the messaging control unit 20 of the messaging device SV trains the message classification model constructed by the message classification model construction processing unit 24 under the control of the message classification model training processing unit 23 in step S43 as follows.

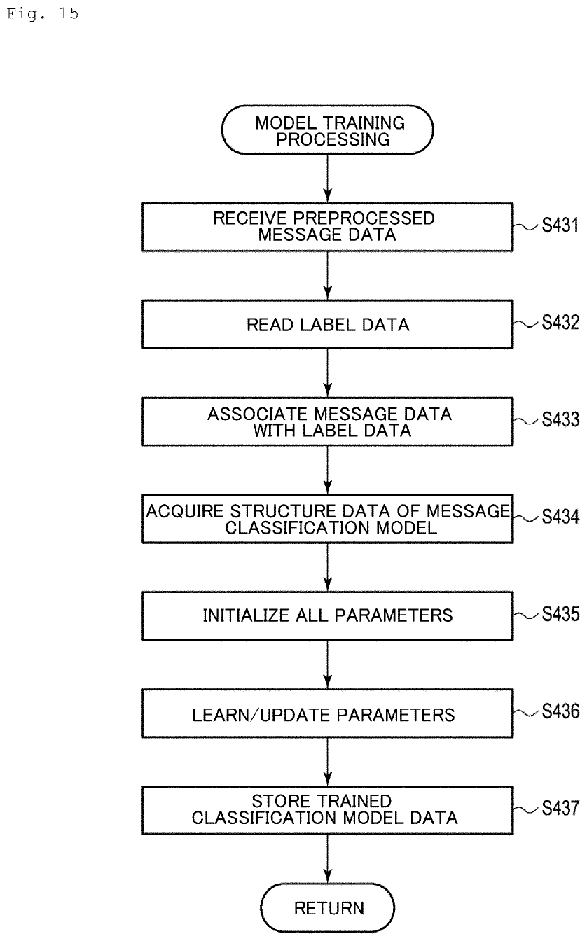

FIG. 15 is a flowchart illustrating an example of a processing procedure and processing details of message classification model training processing executed by the message classification model training processing unit 23.

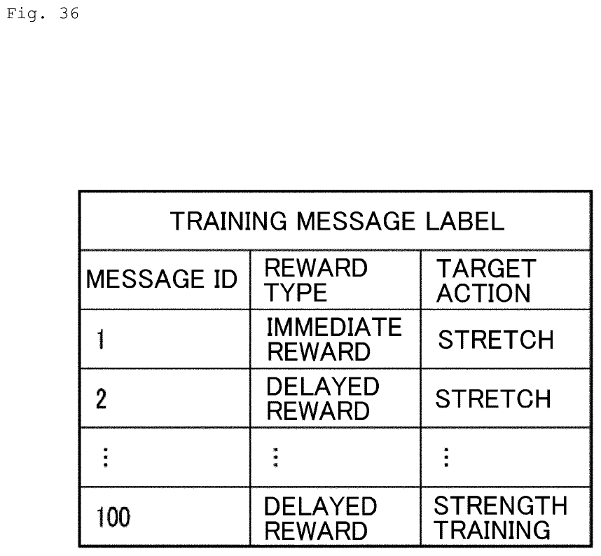

That is, the message classification model training processing unit 23 first receives preprocessed message data from the message data preprocessing unit 22 in step S421. Further, label data is read from the training message label storage unit 43 in step S432. As shown in FIG. 36, for example, label data of a training message is obtained by registering data representing a reward type and a target action in association with a message ID.

Next, the message classification model training processing unit 23 associates the preprocessed message data and the label data which have the same message IDs with each other in step S433. Then, the message classification model training processing unit 23 acquires structure data of the message classification model from the message classification model construction processing unit 24 in step S434.

Next, the message classification model training processing unit 23 initializes parameters of respective layers L1 to L4 of the acquired structure data in step S435. For example, a normally distributed random number with mean 0 and standard deviation 1 is initialized. Then, the parameters are learned and updated using the associated preprocessed message data and label data in step S436. Learning of the parameters can be performed using, for example, a known technique such as a back propagation method based on an error between a value output from the output layer L4 and input label data.

The message classification model training processing unit 23 repeatedly executes the aforementioned model parameter learning/updating processing using all pieces of preprocessed message data and label data corresponding thereto. Then, data representing finally obtained model parameters and model structure is stored in the message classification model storage unit 44 as data representing the trained message classification model in step S437.

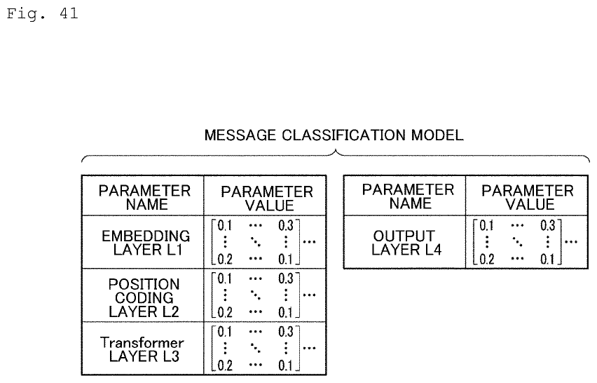

FIG. 41 illustrates an example of a trained message classification model stored in the message classification model storage unit 44.

### (3) Messaging Phase

### (3-1) Estimation of Present Bias

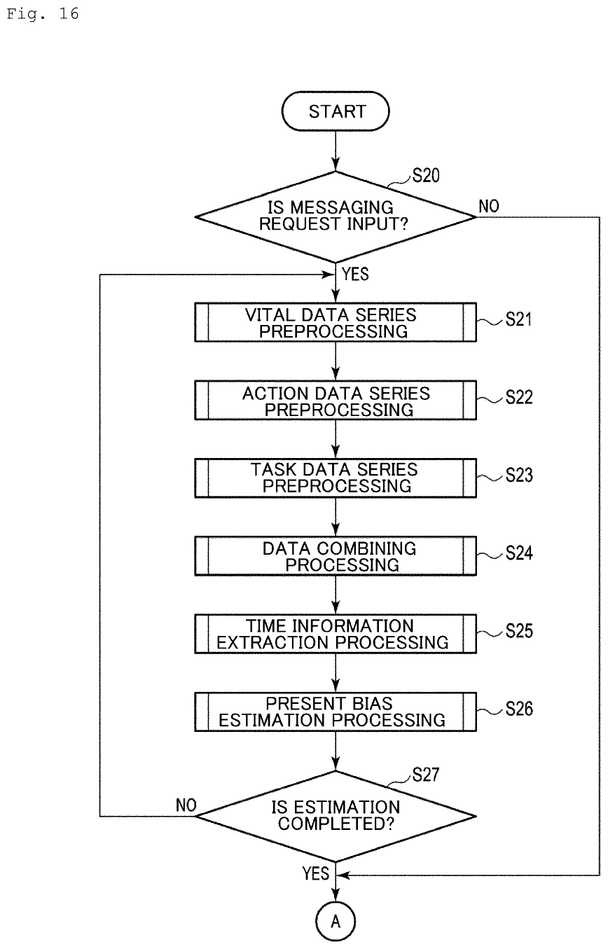

FIG. 16 is a flowchart illustrating an example of a processing procedure and processing details of present bias estimation processing executed by the present bias analysis unit 10 in the control procedure executed by the controller 1 of the messaging device SV in a messaging phase.

For example, it is assumed that a target user or a system manager inputs a messaging request for a certain user to the user terminal UT1 or the manager terminal. Upon detecting input of the messaging request in step S20, the present bias analysis unit 10 of the messaging device SV sets a messaging phase and first executes processing of estimating a present bias of the target user as follows.

### (3-1-1) Acquisition of User Data

When the messaging phase is set, the present bias analysis unit 10 first acquires a vital data series, an action data series, and a task data series of the user in a latest predetermined period, for example, which are transmitted from the user terminal UT1 used by the messaging target user, through the communication I/F unit 4 under the control of the user data acquisition processing unit 18.

### (3-1-2) Preprocessing of Vital Data Series

The present bias analysis unit 10 of the messaging device SV first receives the vital data series of the estimation target user from the user data acquisition processing unit 18 under the control of the vital data preprocessing unit 11 in step S21. Then, as in the case of the present bias learning phase described above, a summary statistic is calculated for each predetermined time interval in accordance with the processing procedure and the processing details shown in FIG. 7 and a preprocessed vital data series including the calculated summary statistic is generated with respect to the received vital data series.

### (3-1-3) Preprocessing of Action Data Series

Next, the controller 1 of the messaging device SV receives the action data series of the estimation target user from the user data acquisition processing unit 18 under the control of the action data preprocessing unit 12 in step S22. Then, as in the case of the present bias learning phase, entries of actions of the estimation target user are aggregated by deleting the entries of duplicate actions and a small number of actions in accordance with the processing procedure and the processing details shown in FIG. 8 to generate a preprocessed action data series with respect to the received action data series.

### (3-1-4) Preprocessing of Task Data

Next, the present bias analysis unit 10 of the messaging device SV receives the task data series of the estimation target user from the user data acquisition processing unit 18 under the control of the task data preprocessing unit 13 in step S23. Then, as in the case of the present bias learning phase, a goal achievement rate is calculated for each entry and a goal progress rate for each user is calculated in accordance with the processing procedure and the processing details shown in FIG. 9 to generate a preprocessed task data series with respect to the received task data series.

### (3-1-5) Data Combining Processing

Then, the present bias analysis unit 10 of the messaging device SV receives the preprocessed vital data series, action data series, and task data series under the control of the data combining processing unit 14 in step S24. Then, as in the case of the present bias learning phase, the preprocessed vital data series, action data series, and task data series are combined by aligning the time positions in accordance with the processing procedure and the processing details shown in FIG. 10, and the combined data is output as feature data of the estimation target user.

### (3-1-6) Extraction of Time Information

Next, the present bias analysis unit 10 of the messaging device SV extracts time information from the combined feature data of the estimation target user and adds the extracted time information to the combined feature data to generate feature data with time information in accordance with the processing procedure and the processing details shown in FIG. 11, as in the case of the present bias learning phase, under the control of the time information extraction processing unit 15 in step S25.

### (3-1-7) Estimation of Present Bias

Next, the present bias analysis unit 10 of the messaging device SV executes processing of estimating the present bias of the messaging target user under the control of the present bias estimation processing unit 19 as described below in step S26.

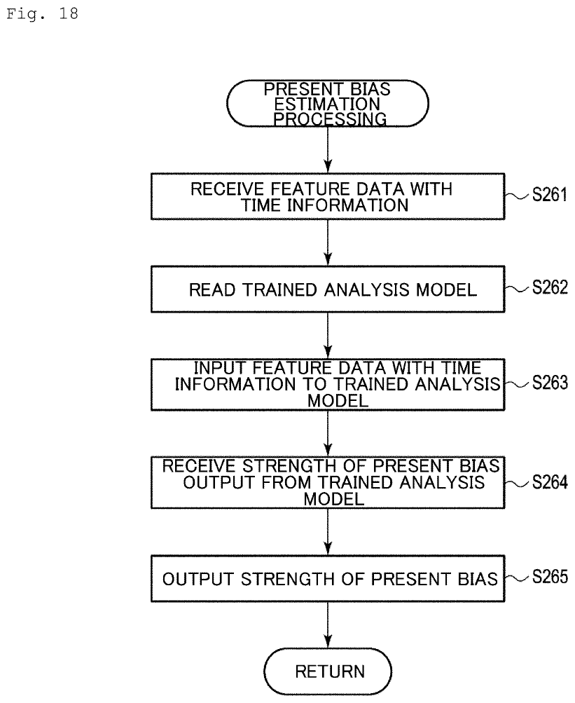

FIG. 18 is a flowchart illustrating an example of a processing procedure and processing details of present bias estimation processing executed by the present bias estimation processing unit 19.

That is, the present bias estimation processing unit 19 first receives the feature data with time information of the messaging target user from the time information extraction processing unit 15 in step S261. Then, the present bias estimation processing unit 19 reads the trained present bias analysis model MD from the analysis model storage unit 35 in step S262, and inputs the feature data with time information of the messaging target user to the read trained present bias analysis model MD in step S263.

As a result, estimation data representing the strength of the present bias of the messaging target user corresponding to the feature data with time information is output from the trained present bias analysis model MD. The present bias estimation processing unit 19 receives the present bias estimation data output from the trained present bias analysis model in step S264, and outputs the received estimation data to the messaging control unit 20 in step S265.

### (3-1-8) Determination of End of Estimation Processing

The present bias analysis unit 10 of the messaging device SV determines whether or not processing of estimating the strengths of present biases with respect to all requested messaging target users ends in step S27. If a target user for which estimation processing is not yet performed remains, processing returns to step S21, and then a series of processing for estimating the present bias of the target user is repeatedly executed in steps S21 to S27.

On the other hand, if processing of estimating the strengths of present biases ends for all messaging target users, the present bias analysis unit 10 of the messaging device SV returns to the standby state.

### (3-2) Messaging Control

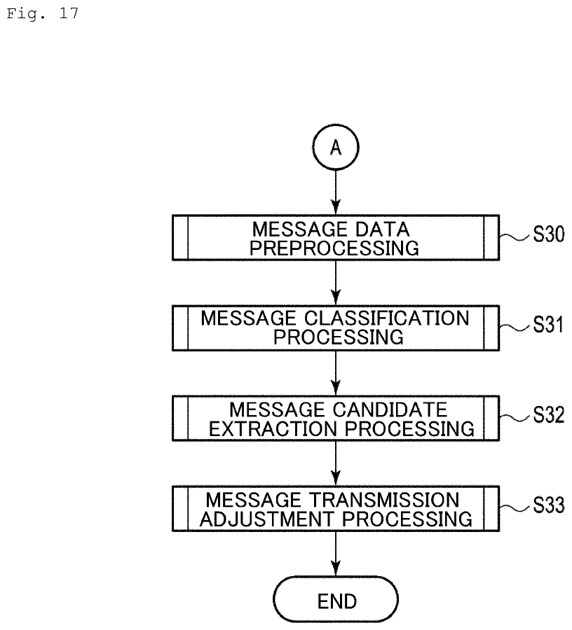

FIG. 17 is a flowchart illustrating an example of a processing procedure and processing details of messaging control processing executed by the messaging control unit 20 in the control procedure executed by the controller 1 of the messaging device SV in a messaging phase.

### (3-2-1) Preprocessing of Message

The messaging control unit 20 first performs preprocessing on each piece of message data included in a message list under the control of the message data preprocessing unit 22 in step S30 as follows.

That is, the message data preprocessing unit 22 first reads a message list to be used in a messaging phase from the message data storage unit 41 in step S411 shown in FIG. 14. Then, as in the case of the message learning phase described above, the message data preprocessing unit 22 decomposes the read message data into words according to the processing procedure and processing details of steps S412 to S416 in FIG. 14, extracts token IDs corresponding to these words from a token dictionary, and generates a token list composed of a list of the extracted token IDs. Then, the generated token list is output as preprocessed message data. When a word is not included in the token dictionary, this word is ignored as a non-processing target.

### (3-2-2) Message Classification Processing

Next, the messaging control unit 20 classifies messages under the control of the message classification processing unit 25 in step S31 as follows.

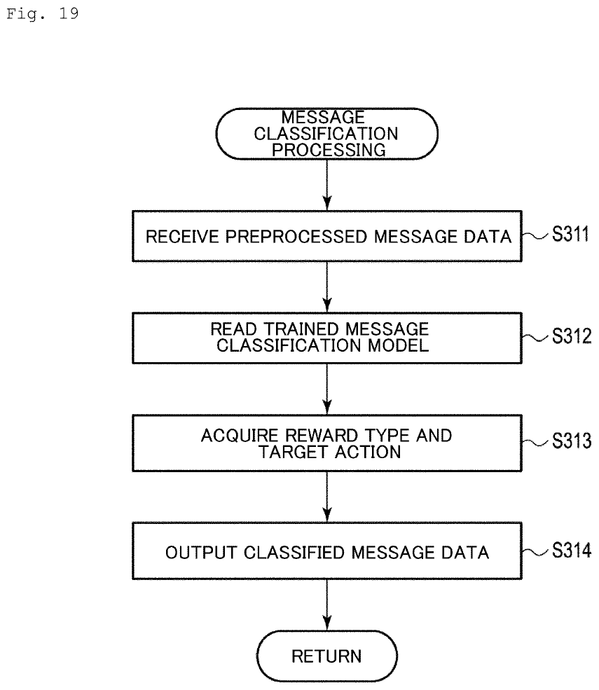

FIG. 19 is a flowchart illustrating an example of a processing procedure and processing details of message classification processing executed by the message classification processing unit 25.

That is, the message classification processing unit 25 first receives preprocessed message data from the message data preprocessing unit 22 in step S311, and further reads a message classification model from the message classification model storage unit 44 in step S312. Then, the message classification processing unit 25 inputs token data included in the preprocessed message data to the message classification model, and acquires data representing reward types and target actions of messages from the message classification model in step S313. Then, the message classification processing unit 25 outputs data representing the reward types and the target actions of the messages to the message candidate extraction processing unit 26 in step S314.

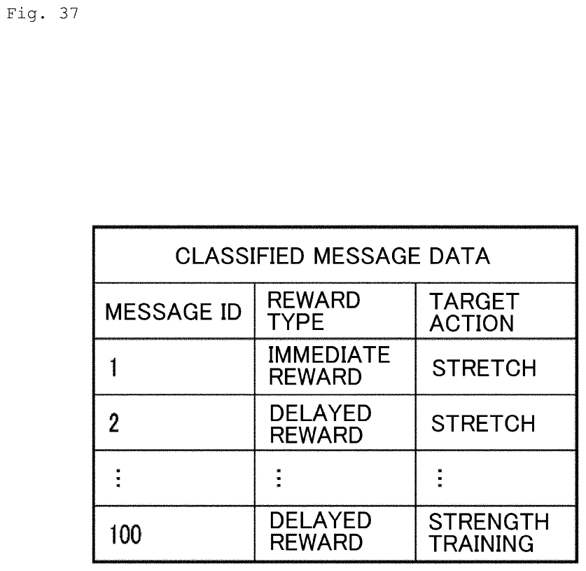

FIG. 37 illustrates an example of data representing reward types and target actions of messages output from the message classification processing unit 25.

### (3-2-3) Extraction of Message Candidates

Next, the messaging control unit 20 extracts message candidates under the control of the message candidate extraction processing unit 26 in step S32 as follows.

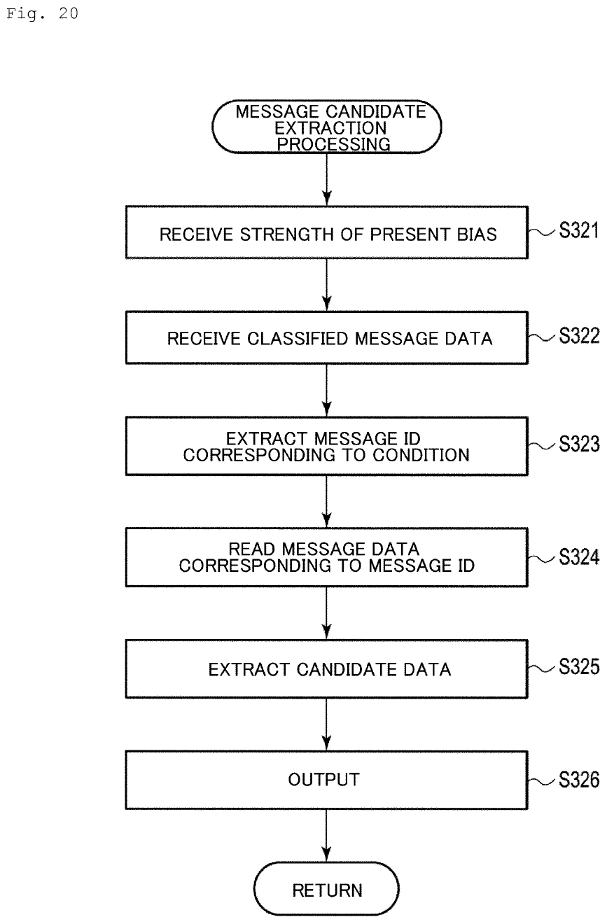

FIG. 20 is a flowchart illustrating an example of a processing procedure and processing details of message candidate extraction processing executed by the message candidate extraction processing unit 26.

That is, the message candidate extraction processing unit 26 first receives estimation data of the strength of a present bias from the present bias analysis unit 10 in step S321, and receives data representing a reward type and a target action of a message from the message classification processing unit 25 in step S322.

Next, the message candidate extraction processing unit 26 refers to the strength of the present bias and the target action, and extracts a message ID corresponding to conditions from classified message data in step S323. For example, when the strength of the present bias is equal to or greater than a preset threshold value, a message ID corresponding to a reward type of “immediate reward” is extracted. On the other hand, when the strength of the present bias is less than the threshold value, a message ID corresponding to a reward type of “delayed reward” is extracted. The threshold value can be arbitrarily set by the system manager.

Subsequently, the message candidate extraction processing unit 26 reads message data corresponding to the message ID from the message data storage unit 41 in step S324. Then, a data table having the message ID, the reward type, the target action, and the message data as attributes is generated in step S325, the generated data table is output as message candidate data in step S326.

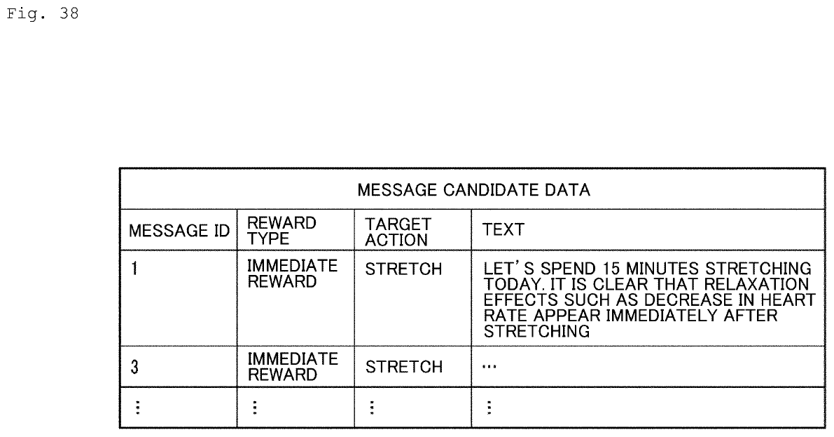

FIG. 38 illustrates an example of the configuration of the message candidate data output from the message candidate extraction processing unit 26 as a result of the above-described message candidate extraction processing.

### (3-2-4) Message Transmission Adjustment Processing

Finally, the messaging control unit 20 executes message transmission processing and processing of updating the number of times of message transmission under the control of the message transmission adjustment processing unit 27 in step S33 as follows.

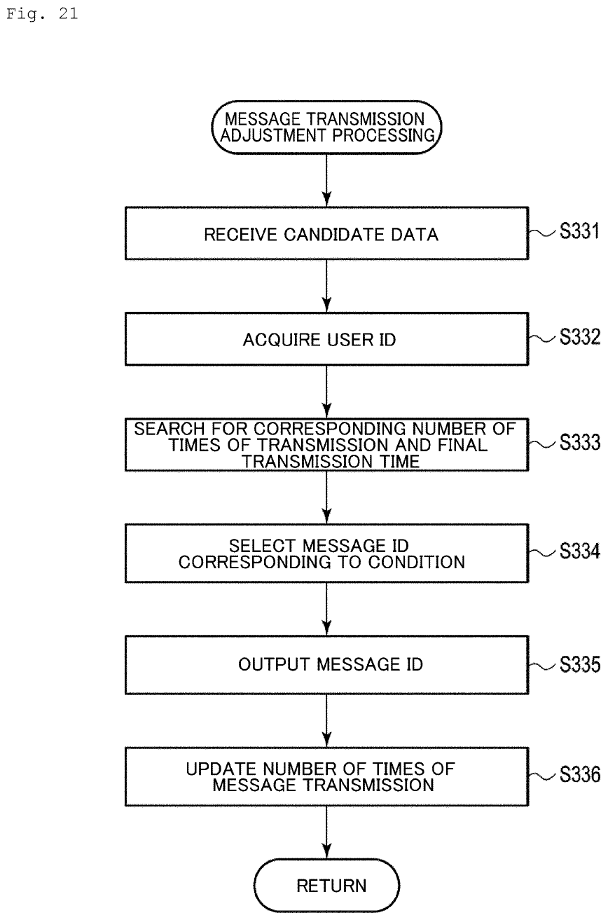

FIG. 21 is a flowchart illustrating an example of a processing procedure and processing details of message transmission adjustment processing executed by the message transmission adjustment processing unit 27.

That is, the message transmission adjustment processing unit 27 first receives message candidate data from the message candidate extraction processing unit 26 in step S331, and further acquires a user ID of a user who is a messaging target in step S332.

Next, the message transmission adjustment processing unit 27 reads the numbers of times of transmission and final transmission times corresponding to the message IDs and user IDs of the candidate data from the message transmission history storage unit 45 in step S333. Then, the message transmission adjustment processing unit 27 selects one message ID corresponding to a preset transmission condition in step S334. Here, as the transmission condition, for example, either a message ID corresponding to a message having the smallest number of times of transmission or a message ID having the largest difference between the present time and the final transmission time is set. The system manager can arbitrarily determine which message ID is to be set.

The message transmission adjustment processing unit 27 reads message data corresponding to the selected message ID from the message data storage unit 41 in step S335. Then, the read message data is transmitted through the communication I/F unit 4 to the user terminal UT1 owned by the messaging target user.

Further, after transmission of the message data, the message transmission adjustment processing unit 27 performs increment processing to increase the number of times of transmission corresponding to the message ID, stored in the message transmission history storage unit 45, by 1, and updates the final transmission time to the latest time in step S336.

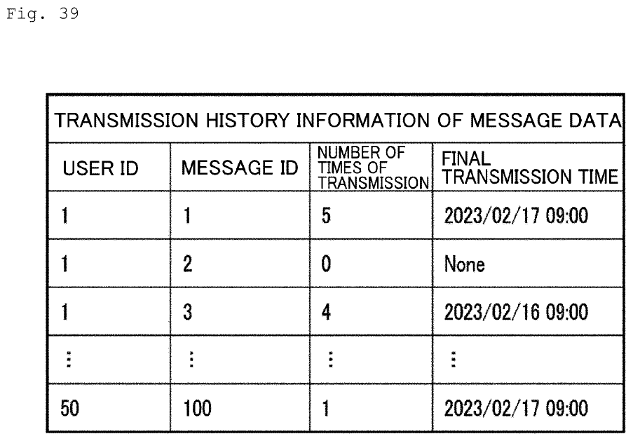

FIG. 39 illustrates an example of the updated number of times of transmission and final transmission time stored in the message transmission history storage unit 45.

### Operations and Effects

As described above, the messaging device SV according to an embodiment as described above provides the following operations and effects.

That is, in a present bias learning phase, the messaging device SV first performs predetermined preprocessing on vital data, action data, and task data of a plurality of users, which have been collected and accumulated in advance, trains a present bias analysis model constructed by a DNN using feature data generated by combining the data with time positions being aligned for each user and known data of the present bias of each user, and stores the trained present bias analysis model MD.

In addition, in a message learning phase, the messaging device SV divides a plurality of pieces of message data stored in advance into tokens through preprocessing to generate a token list, trains a message classification model constructed by a DNN using the token list and label data of a corresponding training message, and stores the message classification model after training processing.

Then, in a messaging phase, the messaging device SV first acquires a vital data series, an action data series, and a task data series of a user who is a messaging target, performs the aforementioned predetermined preprocessing on the acquired data series, generates feature data of the target user by combining the data series with time positions being aligned, and obtains data representing the strength of the present bias of the target user by inputting the generated feature data to the trained present bias analysis model MD.

Next, the messaging device SV inputs a token list of message data included in a message list to the message classification model to classify reward types and target actions of the message data. Then, a reward type corresponding to the target action being executed by the target user and corresponding to the strength of the present bias of the target user is determined, and message data candidates corresponding to the determination result is selected. Further, message data in which a transmission history corresponding to the message ID and the ID of the target user, for example, the number of times of transmission and the final transmission time, satisfies a preset transmission condition is selected from among the requested message data candidates, and the selected message data is transmitted to the user terminal of the target user.

Therefore, according to an embodiment, it is possible to estimate the present bias of the target user by using vital data, action data, and task data obtained by a vital sensor and a scheduler in the daily life of the target user without relying on questionnaire surveys.

Further, according to an embodiment, message data is classified into reward types and target actions, and message data in which a classified target action corresponds to the action being executed by the target user and a reward type corresponds to the strength of the present bias of the target user is selected and presented to the target user. Accordingly, message data in consideration of the action type being executed by the target user and the strength of the present bias can be presented to the target user, and thereby, effective messaging can be performed.

Furthermore, since the message data having the smallest number of times of transmission in the past or the oldest final transmission time is selected at the time of selecting the message data, the problem that the same message data is frequently presented to the target user within a relatively short period of time can be prevented.

### Other Embodiments

(1) In an embodiment, an example of a case in which a server computer disposed on a web or a cloud has the function of the messaging device SV has been described. However, the present invention is not limited thereto, and the functions of the present bias analysis unit 10 and the messaging control unit 20 of the messaging device SV may be distributed to a plurality of server computers capable of mutual communication via a network NW, for example, or may be provided in a user terminal or a manager terminal, for example. In this case, a program for realizing the aforementioned functions of the messaging device SV may be prepared in a server computer, and the user terminal or the manager terminal may download and use the program as necessary.
(2) In an embodiment, an example of a case in which the messaging device SV is provided with the storage units 31 to 33 for storing vital data, action data, and task data for training, and the storage unit 34 for storing present bias data of a learning target user has been described. However, the present invention is not limited thereto, and the present bias analysis data storage unit 30 the storage units 31 to 3, and the messaging control data storage unit 40 may be provided in a data storage server or the like on a cloud, for example, and when the messaging device SV trains the analysis model and the message classification model, training data may be downloaded from the data storage server.
(3) In an embodiment, an example of a case in which the present bias analysis model and the message classification model are constructed using the DNN has been described.

However, the present bias analysis model and the message classification model may be constructed using other neural networks such as a convolution neural network (CNN), a recurrent neural network (RNN), a generative adversarial network (GAN), and the like.
(4) In addition, the functional configuration of the messaging device, the procedure and processing details of processing for executing each function, types of vital data, types and content of action and task, types and configurations of message data, and the like can be modified and implemented in various manners without departing from the scope of the present invention.

Although the embodiments of the present invention have been described in detail above, the above description is merely illustrative of the present invention in every respect. It goes without saying that various modifications and variations can be made without departing from the scope of the present invention. In other words, a specific configuration according to the embodiment may be appropriately employed in implementing the present invention.

In short, the present invention is not limited to the embodiment as it is, and the components can be modified and embodied without departing from the gist of the invention in an implementation stage. Further, various inventions can be formed through appropriate combinations of a plurality of components disclosed in the embodiment. For example, some components of all the components shown in the embodiment may be omitted. Further, constituent elements in different embodiments may be appropriately combined.

### REFERENCE SIGNS LIST

SV Messaging device
UT1 to UTn User terminal
NW Network
1 Controller
1A Present bias analyzer
1B Messaging controller
2 Program storage unit
3 Data storage unit
4 Communication I/F unit
5 Bus
10 Present bias analysis unit
11 Vital data preprocessing unit
12 Action data preprocessing unit
13 Task data preprocessing unit
14 Data combining processing unit
15 Time information extraction processing unit
16 Analysis model training processing unit
17 Analysis model construction processing unit
18 User data acquisition processing unit
19 Present bias estimation processing unit
20 Messaging control unit
21 Message acquisition processing unit
22 Message data preprocessing unit
23 Message classification model training processing unit
24 Message classification model construction processing unit
25 Message classification processing unit
26 Message candidate extraction processing unit
27 Message transmission adjustment processing unit
30 Present bias analysis data storage unit
31 Vital data storage unit
32 Action data storage unit
33 Task data storage unit
34 Present bias data storage unit
35 Analysis model storage unit
40 Messaging control data storage unit
41 Message data storage unit
42 Token storage unit
43 Training message label storage unit
44 Message classification model storage unit
45 Message transmission history storage unit

1. A messaging device comprising:
first processing circuitry configured to analyze a present bias of a target user; and
second processing circuitry configured to perform messaging to the target user,
wherein the first processing circuitry is configured to:
acquire vital data, action data, and task data of a plurality of learning target users over a predetermined period, and perform first preprocessing on the acquired vital data, the acquired action data, and the acquired task data;
generate feature data relating to the plurality of learning target users by combining the preprocessed vital data, the preprocessed action data, and the preprocessed task data for the same learning target user and with positions being aligned in time series; and
a third train a present bias analysis model having a neural network structure constructed in advance by using the feature data relating to the plurality of learning target users and present bias data that is a correct answer to generate a trained present bias analysis model, and
the second processing circuitry is configured to:
acquire a plurality of pieces of training message data, and perform second preprocessing on the acquired training message data to generate a token list constituting the training message data; and
train a message classification model having a neural network structure constructed in advance by using the token list of the plurality of pieces of training message data and preset label data to generate a trained message classification model.

2. The messaging device according to claim 1, wherein the first processing circuitry is further configured to:
acquire a vital data series, an action data series, and a task data series relating to the target user;
perform the first preprocessing on the acquired vital data series, the acquired action data series, and the acquired task data series;
a third generate feature data relating to the target user by combining the vital data series, the action data series, and the task data series on which the first preprocessing has been performed with positions being aligned in time series; and
a fourth input the feature data relating to the target user to the present bias analysis model and to obtain present bias estimation data of the target user from the present bias analysis model, and
the second processing circuitry is further is configured to:
acquire a plurality of pieces of presentation message data, perform the second preprocessing on the acquired presentation message data, and generate a token list constituting the presentation message data;
input the generated token list to the message classification model and obtain data representing rewards and types of target actions corresponding to the presentation message data from the message classification model;
acquire data representing a type of a target action being executed or scheduled to be executed by the target user, and select the presentation message data corresponding to the acquired type of the target action and having a type of reward corresponding to the present bias estimation data; and
output the selected presentation message data to present the selected presentation message data to the target user.

3. The messaging device according to claim 2, wherein the first processing circuitry performs at least one of data transformation processing, aggregation processing, and interpolation processing on the acquired vital data series, the acquired action data series, and the acquired task data series in accordance with characteristics of data to be processed corresponding to the acquired vital data series, the action data series, and the task data series.

4. The messaging device according to claim 2, wherein the first processing circuitry extracts time information represented by a plurality of units from the feature data relating to the target user on the basis of information indicating positions of the vital data series, the action data series, and the task data series in the time series,
assigns the extracted time information to the feature data, and
inputs the feature data to which the time information has been assigned to the present bias analysis model.

5. The messaging device according to claim 2, wherein the second processing circuitry extracts a plurality of candidates for the presentation message data having a type of reward corresponding to the acquired type of the target action of the target user and corresponding to the present bias estimation data, and
selects, as the presentation message data, a candidate whose transmission history satisfies a preset condition from among the plurality of candidates for the presentation message data.

6. A messaging method comprising,
in a learning phase of a present bias analysis model,
acquiring vital data, action data, and task data of a plurality of learning target users over a predetermined period, and performing first preprocessing on the acquired vital data, the acquired action data, and the acquired task data;
generating feature data relating to the plurality of learning target users by combining the preprocessed vital data, the preprocessed action data, and the preprocessed task data for the same learning target user and with positions being aligned in time series; and
training a present bias analysis model having a neural network structure constructed in advance by using the feature data relating to the plurality of learning target users and present bias data that is a correct answer to generate a trained present bias analysis model, and
in a learning phase of a message classification model,
acquiring a plurality of pieces of training message data, and performing second preprocessing on the acquired training message data to generate a token list constituting the training message data; and
training a message classification model having a neural network structure constructed in advance by using the token list of the plurality of pieces of training message data and preset label data to generate a trained message classification model.

7. The messaging method according to claim 6, further comprising, in a messaging phase:
acquiring a vital data series, an action data series, and a task data series relating to a messaging target user;
performing the first preprocessing on the acquired vital data series, the acquired action data series, and the acquired task data series;
generating feature data relating to the target user by combining the vital data series, the action data series, and the task data series on which the first preprocessing has been performed with positions being aligned in time series;
inputting the feature data relating to the target user to the present bias analysis model and obtaining present bias estimation data of the target user from the present bias analysis model;
acquiring a plurality of pieces of presentation message data, performing the second preprocessing on the acquired presentation message data, and generating a token list constituting the presentation message data;
inputting the generated token list to the message classification model and obtaining data representing rewards and types of target actions corresponding to the presentation message data from the message classification model;
acquiring data representing a type of a target action being executed or scheduled to be executed by the target user, and selecting the presentation message data corresponding to the acquired type of the target action and having a type of reward corresponding to the present bias estimation data; and
outputting the selected presentation message data to present the selected presentation message data to the target user.

8. A non-transitory computer readable storage medium storing a computer program which is executed by first processing circuitry and second processing circuitry to provide operations of:
in the first processing circuitry,
analyzing a present bias of a target user;
acquiring vital data, action data, and task data of a plurality of learning target users over a predetermined period, and to perform first preprocessing on the acquired vital data, the acquired action data, and the acquired task data;
generating feature data relating to the plurality of learning target users by combining the preprocessed vital data, the preprocessed action data, and the preprocessed task data for the same learning target user and with positions being aligned in time series; and
training a present bias analysis model having a neural network structure constructed in advance by using the feature data relating to the plurality of learning target users and present bias data that is a correct answer to generate a trained present bias analysis model, and
in the second processing circuitry,
performing messaging to the target user,
acquiring a plurality of pieces of training message data, and performing second preprocessing on the acquired training message data to generate a token list constituting the training message data; and
training a message classification model having a neural network structure constructed in advance by using the token list of the plurality of pieces of training message data and preset label data to generate a trained message classification model.
